(19) United States
(12) Patent Application Publication
(10) Pub. No.: US 2008/0188503 A1
(43) Pub. Date: Aug. 7, 2008

(54) New Compounds II

(75) Inventors: Jeremy Burrows, Sodertalje (SE); Fernando Huerta, Sodertalje (SE); Tobias Rein, Sodertalje (SE); Didier Rotticci, Sodertalje (SE); Karin Staaf, Sodertalje (SE); Dominika Turek, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZenaca AB, Sodertalje (SE)

(21) Appl. No.: 11/769,113

(22) Filed: Jun. 27, 2007

Related U.S. Application Data
(60) Provisional application No. 60/816,755, filed on Jun. 27, 2006.

(51) Int. Cl. A61K 31/506; C07D 401/04; A61P 25/28

(52) U.S. Cl. 514/275; 544/331

(57) ABSTRACT

The present invention relates to a compound of formula (I):

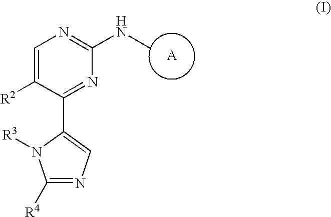

as a free base or a pharmaceutically acceptable salt thereof. The present invention also relates to pharmaceutical formulations containing said compound and to the use of said compound in therapy. The present invention further relates to a process for the preparation of the compound of formula (I).

### TECHNICAL FIELD OF INVENTION

The present invention relates to new compounds of formula (I), as a free base or a pharmaceutically acceptable salt thereof, to pharmaceutical formulations containing said compounds and to the use of said compounds in therapy. The present invention further relates to a process for the preparation of compounds of formula (I) and to new intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but is believed to be related. Glycogen synthase kinase 3β (GSK3β) or Tau phosphorylating kinase selectively phosphorylates the microtubule associated protein Tau in neurons at sites that are hyperphosphorylated in AD brains. Hyperphosphorylated tau has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of tau and a paired helical filaments-like state via induction of GSK3β activity, followed by disruption of axonal transport and neuronal death (Imahori and Uchida, J. Biochem. 1997, 121:179-188). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 1996, 93: 2719-2723). Acetyl-Co-A is critical for the synthesis of acetylcholine, a neurotransmitter with cognitive functions. Accumulation of amyloid-β is an early event in AD. GSK Tg mice show increased levels of amyloid-β in brain. Also, PDAPP mice fed with Lithium show decreased amyloid-β levels in hippocampus and decreased amyloid plaque area (Su et al., Biochemistry 2004, 43: 6899-6908). Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3β inhibition. Recent studies (Bhat et. al., PNAS 2000, 97: 11074-11079) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as cognitive disorders, Alzheimer's Disease, Parkinson's Disease, amyotrophic lateral sclerosis, Huntington's Disease and HIV dementia and traumatic brain injury; and as in ischemic stroke. Lithium was neuroprotective in inhibiting apoptosis in cells and in the brain at doses that resulted in the inhibition of GSK3β. Thus GSK3β inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The discovery that lithium inhibits GSK3 at therapeutic concentrations has raised the possibility that this enzyme represents a key target of lithium's action in the brain (Stambolic et al., Curr. Biol. 1996, 68(12):1664-1668, 1996; Klein and Melton; PNAS 1996, 93:8455-8459; Gould et al., Neuropsychopharmacology, 2005, 30:1223-1237). GSK3 inhibitor has been shown to reduce immobilisation time in forced swim test, a model to assess on depressive behavior (O'Brien et al., J Neurosci 2004, 24(30): 6791-6798). GSK3 has been associated with a polymorphism found in bipolar II disorder (Szczepankiewicz et al., Neuropsychobiology. 2006, 53: 51-56). Inhibition of GSK3β may therefore be of therapeutic relevance in the treatment of BD as well as in AD patients that have affective disorders.

### Schizophrenia

Accumulating evidence implicates abnormal activity of GSK3 in mood disorders and schizophrenia. GSK3 is involved in signal transduction cascades of multiple cellular processes, particularly during neural development. (Kozlovsky et al., Am. J. Psychiatry, 2000, 157, 5: 831-833) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 1998, 9(7):1379-1383). Atypical antipsychotic such as olanzapine, clozapine, quetiapine, and ziprasidone, inhibits GSK3 by increasing ser9 phosphorylation suggesting that antipsychotics may exert their beneficial effects via GSK3 inhibition (Li X. et al., Int. J. of Neuropsychopharmacol, 2007, 10: 7-19, Epubl. 2006, May 4).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2): 263-71). Inhibition of GSK3 increases the activity of glycogen synthase thereby decreasing glucose levels by its conversion to glycogen. In animal models of diabetes, GSK3 inhibitors lowered plasma glucose levels up to 50% (Cline et al., Diabetes, 2002, 51: 2903-2910; Ring et al., Diabetes 2003, 52: 588-595). GSK3 inhibition may therefore be of therapeutic relevance in the treatment of Type I and Type II diabetes and diabetic neuropathy.

### Alopecia

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell, 1998, 95(5): 605-14)). The new follicles formed sebaceous glands and dermal papilla, normally established only in embryogenesis. Thus GSK3 inhibition may offer treatment for baldness.

### Inflammatory Disease

The discovery that GSK3 inhibitors provide anti-inflammatory effects has raised the possibility of using GSK3 inhibitors for therapeutic intervention in inflammatory diseases. (Martin et al., Nat. Immunol. 2005, 6(8): 777-784; Jope et al., Neurochem. Res. 2006, DOI 10.1007/s11064-006-9128-5)). Inflammation is a common feature of a broad range of conditions including Alzheimer's Disease and mood disorders.

### Cancer

GSK3 is overexpressed in ovarian, breast and prostate cancer cells and recent data suggests that GSK3b may have a role in contributing to cell proliferation and survival pathways in several solid tumor types. GSK3 plays an important role in several signal transduction systems which influence cell proliferation and survival such as WNT, PI3 Kinase and NFkB. GSK3b deficient MEFs indicate a crucial role in cell survival mediated NFkB pathway (Ougolkov A V and Billadeau D D., Future Oncol. 2006 February; 2(1): 91-100.). Thus, GSK3 inhibitors may inhibit growth and survival of solid tumors, including pancreatic, colon and prostate cancer.

### Bone-Related Disorders and Conditions

It has been shown that GSK3 inhibitors could be used for treatment of bone-related disorders. This has been discussed in e.g. Tobias et al., Expert Opinion on Therapeutic Targets, February 2002, pp 41-56.GSK3 inhibitors could be used for treatment of bone-related disorders or other conditions, which involves a need for new and increased bone formation. Remodeling of the skeleton is a continuous process, controlled by systemic hormones such as parathyroid hormone (PTH), local factors (e.g. prostaglandin E2), cytokines and other biologically active substances. Two cell types are of key importance: osteoblasts (responsible for bone formation) and osteoclasts (responsible for bone resorption). Via the RANK, RANK ligand and osteoprotegerin regulatory system these two cell types interact to maintain normal bone turnover (Bell N H, Current Drug Targets—Immune, Endocrine & Metabolic Disorders, 2001, 1:93-102).

Osteoporosis is a skeletal disorder in which low bone mass and deterioration of bone microarchitecture lead to increased bone fragility and fracture risk. To treat osteoporosis, the two main strategies are to either inhibit bone resorption or to stimulate bone formation. The majority of drugs currently on the market for the treatment of osteoporosis act to increase bone mass by inhibiting osteoclastic bone resorption. It is recognized that a drug with the capacity to increase bone formation would be of great value in the treatment of osteoporosis as well as having the potential to enhance fracture healing in patients.

Recent in vitro studies suggest a role of GSK3β in osteoblast differentiation. First, it has been shown that glucocorticoids inhibit cell cycle progression during osteoblast differentiation in culture. The mechanism behind this is activation of GSK3β in osteoblasts, resulting in c-Myc down-regulation and impediment of the G₁/S cell cycle transition. The attenuated cell cycle and reduced c-Myc level are returned to normal when GSK3β is inhibited using lithium chloride (Smith et al., J. Biol. Chem., 2002, 277: 18191-18197). Secondly, inhibition of GSK3β in the pluripotent mesenchymal cell line C3H10T1/2 leads to a significant increase in endogenous β-catenin signaling activity. This, in turn, induces expression of alkaline phosphatase mRNA and protein, a marker of early osteoblast differentiation (Bain et al., Biochem. Biophys. Res. Commun., 2003, 301: 84-91).

### DISCLOSURE OF THE INVENTION

The present invention provides a compound of formula (I):

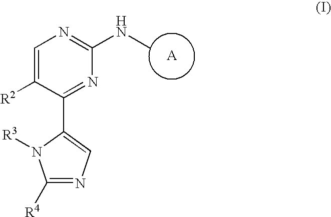

wherein:
A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by a group —R⁵—R⁷, with the proviso that said carbocyclyl is not phenyl;
R¹ is selected from halo, nitro, cyano, hydroxy, amino, sulphamoyl, carbamoyl, C₁₋₃alkyl, a carbocyclyl, a heterocyclyl and a group —R⁶—R⁷, wherein said C₁₋₃alkyl is optionally substituted by one or more halo and wherein said carbocyclyl or heterocyclyl optionally forms a conjugated ring system together with A;
R² is selected from halo, nitro, trifluoromethyl, trifluoromethoxy and cyano;
R³ is selected from methyl, C₆alkyl, C₆alkenyl, C₆alkynyl, a 6-membered non-aromatic carbocyclyl and a 6-membered non-aromatic heterocyclyl, wherein said C₆alkyl, C₆alkenyl, C₆alkynyl, carbocyclyl or heterocyclyl is optionally substituted by one or more halo, cyano, trifluoromethoxy, C₁₋₃haloalkyl or C₁₋₃alkyl;
R⁴ is selected from hydrogen, C₁₋₃alkyl, cyano and C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl;
R⁵ is selected from —C(O)N(R⁹)—, —S(O)_{Z}—, —SO₂N(R¹⁰)—, —SO₂O—, —C(O)—, —C(O)O— and (—CH₂—)ₘ; wherein R⁹ and R¹⁰ are independently selected from hydrogen or C₁₋₆alkyl and wherein said C₁₋₆alkyl is optionally substituted by one or more R¹⁹; and wherein m is 0, 1, 2 or 3 and wherein z is 1 or 2;
R⁶ is selected from —O—, —N(R¹¹)C(O)—, —C(O)N(R¹²)—, —S(O)ᵣ—, —SO₂N(R¹³)—, —N(R¹⁴)SO₂—, —(CH₂)ₚN(R¹⁵)—, —OSO₂—, —C(O)—, —C(O)O—, —N(R¹⁶)C(O)O—, —N(R¹⁷)C(O)N(R¹⁸)—, and (—CH₂—)ₙ; wherein R¹¹, R¹², R¹³, R¹⁴, R¹⁵, R¹⁶, R¹⁷ and R¹⁸ are independently selected from hydrogen or C₁₋₆alkyl and wherein said C₁₋₆alkyl is optionally substituted by one or more R¹⁹; and wherein n is 0, 1, 2 or 3 and wherein p is 0, 1, 2 or 3 and wherein r is 0, 1 or 2;
R⁷ is selected from hydrogen, C₁₋₆alkyl, C₂₋₆alkenyl, C₂₋₆alkynyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹;
R¹⁹ and R²⁰ are independently selected from halo, nitro, cyano, hydroxy, amino, carboxy, carbamoyl, sulphamoyl, C₁₋₆alkyl, C₂₋₆alkenyl, C₂₋₆alkynyl, C₁₋₆alkoxy, C₁₋₆alkoxyC₁₋₆alkoxy, C₁₋₆alkanoyl, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R²²—, heterocyclylC₁₋₆alkyl-R²³—, carbocyclyl-R²⁴— and heterocyclyl-R²⁵—; wherein a is 0, 1 or 2; and wherein R¹⁹ and R²⁰ independently of each other is optionally substituted on carbon by one or more R²⁶; and wherein if said heterocyclyl contains an —NH— moiety that nitrogen is optionally substituted by a group selected from R²⁷;
R²², R²³, R²⁴ and R²⁵ are independently selected from —O—, —N(R²⁸)—, —C(O)—, —N(R²⁹)C(O)—, —C(O)N(R³⁰)—, —S(O)_{S}—, —SO₂N(R³¹)— and —N(R³²)SO₂—; wherein R²⁸, R²⁹, R³⁰, R³¹ and R³² are independently selected from hydrogen or C₁₋₆alkyl and s is 0, 1 or 2;
R²¹ and R²⁷ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, carbocyclyl, heterocyclyl, —C₁₋₆alkylcarbocylyl, —C₁₋₆alkylheterocyclyl, benzyloxycarbonyl, benzoyl and phenylsulphonyl; wherein R²¹ and R²⁷ independently of each other is optionally substituted on carbon by one or more R³³; and
R²⁶ and R³³ are independently selected from halo, nitro, cyano, —C₁₋₃alkylhydroxy, —C₁₋₃alkylmethoxy, —C₁₋₃alkylethoxy, —C₁₋₃alkylisopropoxy, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, carbocycle and heterocycle; wherein said carbocycle or heterocycle is optionally substituted by halo, methyl, trifluoromethyl, cyano or ethyl;
as a free base or a pharmaceutically acceptable salt thereof.

One aspect of the present invention relates to a compound of formula (I), wherein A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by —R⁵—R⁷, with the proviso that said carbocycle is not phenyl;

R¹ is selected from halo, nitro, cyano, hydroxy, amino, sulphamoyl, carbamoyl, C₁₋₃alkyl, a carbocyclyl, a heterocyclyl and a group —R⁶—R⁷, wherein said C₁₋₃alkyl is optionally substituted by one or more halo and wherein said carbocyclyl or heterocyclyl optionally forms a conjugated ring system together with A;
R² is selected from halo, trifluoromethyl, trifluoromethoxy and cyano;
R³ is selected from methyl, C₆alkyl, C₆alkenyl, C₆alkynyl, a 6-membered non-aromatic carbocyclyl and a 6-membered non-aromatic heterocyclyl, wherein said C₆alkyl, C₆alkenyl, C₆alkynyl, carbocyclyl or heterocyclyl is optionally substituted by one or more halo, cyano, trifluoromethoxy, C₁₋₃haloalkyl or C₁₋₃alkyl;
R⁴ is selected from hydrogen, C₁₋₃alkyl, cyano and C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl;
R⁵ is selected from —C(O)N(R⁹)—, —S(O)_{Z}—, —SO₂N(R¹⁰)—, —SO₂O—, —C(O)—, —C(O)O— and (—CH₂—)ₘ; wherein R⁹ and R¹⁰ are independently selected from hydrogen or C₁₋₆alkyl and wherein said C₁₋₆alkyl is optionally substituted by one or more R¹⁹; and wherein m is 0, 1, 2 or 3 and wherein z is 1 or 2;
R⁶ is selected from —O—, —N(R¹¹)C(O)—, —C(O)N(R¹²)—, —S(O)ᵣ—, —SO₂N(R¹³)—, —N(R¹⁴)SO₂—, —(CH₂)ₚN(R¹⁵)—, —OSO₂—, —C(O)—, —C(O)O—, —N(R¹⁶)C(O)O—, —N(R¹⁷)C(O)N(R¹⁸)—, and (—CH₂—)ₙ; wherein R¹¹, R¹², R¹³, R¹⁴, R¹⁵, R¹⁶, R¹⁷ and R¹⁸ are independently selected from hydrogen or C₁₋₆alkyl and wherein said C₁₋₆alkyl is optionally substituted by one or more R¹⁹; wherein n is 0, 1, 2 or 3 and wherein p is 0, 1, 2 or 3 and wherein r is 0, 1 or 2;
R⁷ is selected from hydrogen, C₁₋₆alkyl, C₂₋₆alkenyl, C₂₋₆alkynyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹;
R¹⁹ and R²⁰ are independently selected from halo, nitro, cyano, hydroxy, amino, carboxy, C₁₋₆alkyl, C₂₋₆alkenyl, C₂₋₆alkynyl, C₁₋₆alkoxy, C₁₋₆alkoxyC₁₋₆alkoxy, C₁₋₆alkanoyl, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ, carbocyclyl, heterocyclyl, carbocyclylC₁₋₆alkyl-R²²—, heterocyclylC₁₋₆alkyl-R²³—, carbocyclyl-R²⁴— and heterocyclyl-R²⁵—; wherein a is 0, 1 or 2; and wherein R¹⁹ and R²⁰ independently of each other is optionally substituted on carbon by one or more R²⁶; and wherein if said heterocyclyl contains an —NH— moiety that nitrogen is optionally substituted by a group selected from R²⁷;
R²², R²³, R²⁴ and R²⁵ are independently selected from —O—, —N(R²⁸)—, —C(O)—, —N(R²⁹)C(O)—, —C(O)N(R³⁰)—, —S(O)_{S}—, —SO₂N(R³¹)— and —N(R³²)SO₂—; wherein R²⁸, R²⁹, R³⁰, R³¹ and R³² are independently selected from hydrogen or C₁₋₆alkyl and s is 0, 1 or 2;
R²¹ and R²⁷ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, carbocyclyl, heterocyclyl, —C₁₋₆alkylcarbocylyl, —C₁₋₆alkylheterocyclyl, benzyloxycarbonyl, benzoyl and phenylsulphonyl; wherein R²¹ and R²⁷ independently of each other is optionally substituted on carbon by one or more R³³; and
R²⁶ and R³³ are independently selected from halo, nitro, cyano, —C₁₋₃alkylhydroxy, —C₁₋₃alkylmethoxy, —C₁₋₃alkylethoxy, —C₁₋₃alkylisopropoxy, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, methylthio, ethylthio, methylsulphinyl, ethylsulphinyl, is mesyl, ethylsulphonyl, methoxycarbonyl, ethoxycarbonyl, N,N-dimethylsulphamoyl, N,N-diethylsulphamoyl, N-methyl-N-ethylsulphamoyl, carbocycle and heterocycle;
wherein said carbocycle or heterocycle is optionally substituted by halo, methyl, trifluoromethyl, cyano or ethyl.

Another aspect of the present invention relates to a compound of formula (I), wherein A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by —R⁵—R⁷ with the proviso that said carbocyclyl is not phenyl;

R¹ is selected from C₁₋₃alkyl, a carbocyclyl, a heterocyclyl and a group —R⁶—R⁷, wherein said C₁₋₃alkyl is optionally substituted by one or more halo and wherein said carbocyclyl or heterocyclyl optionally forms a conjugated ring system together with A;
R² is selected from halo, trifluoromethyl, trifluoromethoxy and cyano;
R³ is selected from methyl, C₆alkyl, a 6-membered non-aromatic carbocyclyl and a 6-membered non-aromatic heterocyclyl, wherein said C₆alkyl, carbocyclyl or heterocyclyl is optionally substituted by one or more halo, cyano, trifluoromethoxy, C₁₋₃haloalkyl or C₁₋₃alkyl;
R⁴ is selected from hydrogen, C₁₋₃alkyl, cyano and C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl;
R⁵ is selected from —C(O)N(R⁹)—, —S(O)_{Z}—, —SO₂N(R¹⁰)—, —SO₂O—, —C(O)—, —C(O)O— and (—CH₂—)ₘ; wherein R⁹ and R¹⁰ are independently selected from hydrogen or C₁₋₆alkyl and wherein said C₁₋₆alkyl is optionally substituted by one or more R¹⁹; and wherein m is 0, 1, 2 or 3 and wherein z is 1 or 2;
R⁶ is selected from —O—, —N(R¹¹)C(O)—, —C(O)N(R¹²)—, —S(O)ᵣ—, —SO₂N(R¹³)—, —N(R¹⁴)SO₂—, —(CH₂)ₚN(R¹⁵)—, —OSO₂—, —C(O)—, —C(O)O—, —N(R¹⁶)C(O)O—, —N(R¹⁷)C(O)N(R¹⁸)—, and (—CH₂—)ₙ; wherein R¹¹, R¹², R¹³, R¹⁴, R¹⁵, R¹⁶, R¹⁷ and R¹⁸ are independently selected from hydrogen or C₁₋₆alkyl and wherein said C₁₋₆alkyl is optionally substituted by one or more R¹⁹; and wherein n is 0, 1, 2 or 3 and wherein p is 0, 1, 2 or 3 and wherein r is 0, 1 or 2;
R⁷ is selected from hydrogen, C₁₋₆alkyl, C₂₋₆alkenyl, C₂₋₆alkynyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹;
R¹⁹ and R²⁰ are independently selected from halo, nitro, cyano, hydroxy, amino, carboxy, C₁₋₆alkyl, C₂₋₆alkenyl, C₂₋₆alkynyl, C₁₋₆alkoxy, C₁₋₆alkoxyC₁₋₆alkoxy, C₁₋₆alkanoyl, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, carbocyclyl, heterocyclyl, carbocyclylC₁₋₆alkyl-R²²—, heterocyclylC₁₋₆alkyl-R²³—, carbocyclyl-R²⁴— and heterocyclyl-R²⁵—; and wherein R¹⁹ and
R²⁰ independently of each other is optionally substituted on carbon by one or more R²⁶;
and wherein if said heterocyclyl contains an —NH— moiety that nitrogen is optionally substituted by a group selected from R²⁷;
R²², R²³, R²⁴ and R²⁵ are independently selected from —O—, —N(R²⁸)—, —C(O)—, —N(R²⁹)C(O)—, —C(O)N(R³⁰)—, —S(O)_{S}—, —SO₂N(R³¹)— and —N(R³²)SO₂—; wherein R²⁸, R²⁹, R³⁰, R³¹ and R³² are independently selected from hydrogen or C₁₋₆alkyl and s is 0, 1 or 2;
R²¹ and R²⁷ are independently selected from C₁₋₆alkyl, C₁₋₆alkanoyl, C₁₋₆alkoxycarbonyl, carbamoyl, N—(C₁₋₆alkyl)carbamoyl, N,N—(C₁₋₆alkyl)carbamoyl, carbocyclyl, heterocyclyl, —C₁₋₆alkylcarbocylyl, —C₁₋₆alkylheterocyclyl, benzoyl and phenylsulphonyl; wherein R²¹ and
R²⁷ independently of each other is optionally substituted on carbon by one or more R³³; and
R²⁶ and R³³ are independently selected from halo, nitro, cyano, —C₁₋₃alkylhydroxy, —C₁₋₃alkylmethoxy, —C₁₋₃alkylethoxy, —C₁₋₃alkylisopropoxy, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, methylthio, ethylthio, methylsulphinyl, mesyl, ethylsulphonyl, methoxycarbonyl, ethoxycarbonyl, N,N-diethylsulphamoylcarbocycle and heterocycle; wherein said carbocycle or heterocycle is optionally substituted by halo, methyl, trifluoromethyl, cyano or ethyl.

Yet another aspect of the present invention relates to a compound of formula (I), wherein R² is halo or cyano.

A further aspect of the present invention relates to a compound of formula (I), wherein R² is halo. According to one embodiment of the present invention, R² is fluoro.

One aspect of the present invention relates to a compound of formula (I), wherein R³ is selected from a 6-membered non-aromatic carbocyclyl or a 6-membered non-aromatic heterocyclyl, wherein said carbocyclyl or heterocyclyl is optionally substituted by one or more halo, cyano, trifluoromethoxy, C₁₋₃haloalkyl or C₁₋₃alkyl.

Another aspect of the present invention relates to a compound of formula (I), wherein R³ is a non-aromatic 6-membered heterocyclyl.

Yet another aspect of the present invention relates to a compound of formula (I), wherein R³ is 3-tetrahydropyranyl or 4-tetrahydropyranyl.

One aspect of the present invention relates to a compound of formula (I), wherein R³ is 4-tetrahydropyranyl.

Yet one aspect of the present invention relates to a compound of formula (I), wherein R⁴ is C₁₋₃alkyl or C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl.

A further aspect of the present invention relates to a compound of formula (I), wherein R⁴ is C₁₋₃alkyl.

One aspect of the present invention relates to a compound of formula (I), wherein R⁴ is methyl.

Another aspect of the present invention relates to a compound of formula (I), wherein A is heterocyclyl; wherein said heterocyclyl is optionally substituted on carbon by one or more R¹ and wherein if said heterocyclyl contains an —NH— moiety that nitrogen may be optionally substituted by —R⁵—R⁷. According to one embodiment of the present invention, A is 4-piperidinyl, 4-tetrahydropyranyl, 3-pyridyl, 4-pyridyl, 5-pyrimidinyl, 4-isoquinolinyl or 2-pyridyl.

Yet another aspect of the present invention relates to a compound of formula, wherein A is a non-aromatic carbocyclyl; wherein said carbocyclyl is optionally substituted on carbon by one or more R¹. According to one embodiment of the present invention, said non-aromatic carbocyclyl is cyclohexyl.

One aspect of the present invention relates to a compound of formula (I), wherein R¹ is C₁₋₃alkyl, wherein said C₁₋₃alkyl may be optionally substituted by one or more halo. According to one embodiment of the present invention, R¹ is methyl. According to one embodiment of the present invention, R¹ is C₁₋₃alkyl substituted by one or more halo. According to another embodiment of the present invention, R¹ is trifluoromethyl.

Another aspect of the present invention relates to a compound of formula (I), wherein R¹ is selected from a group —R⁶—R⁷. According to one embodiment of the present invention, R⁶ is selected from —O—, —(CH₂)ₚN(R¹⁵)—, —C(O)—, —C(O)O—, —N(R¹⁶)C(O)O—, and (—CH₂—)ₙ. According to another embodiment of the present invention, R⁶ is selected from —O—, —(CH₂)ₚN(R¹⁵)—, —C(O)— and (—CH₂—)ₙ. According to another embodiment of the present invention, R⁶ is (—CH₂—)ₙ and n is 0 or 1. According to another embodiment of the present invention, R⁶ is —(CH₂)ₚN(R¹⁵)— and p is 1.

A further aspect of the present invention relates to a compound of formula (I), wherein R⁵ is selected from —C(O)N(R⁹)—, —S(O)_{Z}—, —C(O)—, —C(O)O— and (—CH₂—)ₘ; and wherein m is 0 or 1 and wherein z is 2. According to one embodiment of the present invention, R⁵ is selected from, —S(O)_{Z}—, —C(O)—, —C(O)O— and (—CH₂—)ₘ; and wherein m is 0 or 1 and wherein z is 2.

According to one embodiment of the present invention, R⁷ is selected from hydrogen, C₁₋₆alkyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹. According to another embodiment of the present invention, R⁷ is C₁₋₆alkyl, heterocyclyl or carbocyclyl; wherein R⁷ may be optionally substituted on carbon by one or more R²⁰; and wherein if said heterocyclyl contains an —NH— moiety that nitrogen may be optionally substituted by a group selected from R²¹. According to yet another embodiment of the present invention, R⁷ is C₁₋₆alkyl. According to a further embodiment of the present invention, R⁷ is methyl.

According to one embodiment of the present invention, A is not substituted.

Another aspect of the present invention relates to a compound of formula (I), wherein A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by a group —R⁵—R⁷, with the proviso that said carbocyclyl is not phenyl;

R¹ is selected from C₁₋₃alkyl, a carbocyclyl, and a group —R⁶—R⁷, wherein said C₁₋₃alkyl is optionally substituted by one or more halo; R² is halo; R³ is a 6-membered non-aromatic heterocyclyl; R⁴ is C₁₋₃alkyl; R⁵ is selected from —S(O)_{Z}—, —C(O)—, —C(O)O— and (—CH₂—)ₘ;
and wherein m is 0 or 1 and wherein z is 2; R⁶ is selected from —O—, —(CH₂)ₚN(R¹⁵)—, —C(O)—, and (—CH₂—)ₙ; wherein R¹⁵ is selected from hydrogen or C₁₋₆alkyl and wherein said C₁₋₆alkyl is optionally substituted by one or more R¹⁹; and wherein n is 0 or 1 and wherein p is 1; R⁷ is selected from hydrogen, C₁₋₆alkyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹; R¹⁹ and R²⁰ are independently selected from halo, cyano, C₁₋₆alkyl, C₁₋₆alkoxy, N—(C₁₋₆alkyl)amino, N,N—(C₁₋₆alkyl)₂amino, carbocyclyl and heterocyclyl; and wherein R¹⁹ and R²⁰ independently of each other is optionally substituted on carbon by one or more R²⁶; R²¹ is C₁₋₆alkanoyl or heterocyclyl; and
R²⁶ is selected from halo, cyano, —C₁₋₃alkylmethoxy, hydroxy, methyl, heterocycle and methoxy; wherein said carbocycle or heterocycle is optionally substituted by halo.

According to one embodiment of the present invention, R² is fluoro. According to another embodiment of the present invention, R³ is 4-tetrahydropyranyl. According to another embodiment of the present invention, R⁴ is methyl.

Yet another aspect of the present invention relates to a compound of formula (I), wherein A is heterocyclyl wherein said heterocyclyl is optionally substituted, on carbon, by one or more R¹; R¹ is C₁₋₃alkyl or a group —R⁶—R⁷, wherein said C₁₋₃alkyl may be optionally substituted by one or more halo; R² is halo; R³ is a 6-membered non-aromatic heterocyclyl;

R⁴ is C₁₋₃alkyl; R⁶ is —O—, or —C(O)—; and R⁷ is C₁₋₆alkyl.

The present invention also provides a compound selected from:
5-Fluoro-4-[2-methyl-1-(tetrahydro-2H-pyran-4-yl)-1H-imidazol-5-yl]-N-pyrimidin-5-ylpyrimidin-2-amine;
1-[5-({5-Fluoro-4-[2-methyl-1-(tetrahydro-2H-pyran-4-yl)-1H-imidazol-5-yl]pyrimidin-2-yl}amino)pyridin-3-yl]ethanone;
5-Fluoro-N-(6-methoxypyridin-2-yl)-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[5-(trifluoromethyl)pyridin-2-yl]pyrimidin-2-amine;
5-Fluoro-N-(6-methylpyridin-3-yl)-4-[2-methyl-1-(tetrahydro-2H-pyran-4-yl)-1H-imidazol-5-yl]pyrimidin-2-amine;
5-Fluoro-N-(4-methoxypyridin-2-yl)-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[6-(morpholin-4-ylmethyl)pyridin-3-yl]pyrimidin-2-amine;
5-Fluoro-4-[2-methyl-1-(tetrahydro-2H-pyran-4-yl)-1H-imidazol-5-yl]-N-[6-(piperidin-1-ylmethyl)pyridin-3-yl]pyrimidin-2-amine;
5-Fluoro-N-{6-[(4-methyl-1,4-diazepan-1-yl)methyl]pyridin-3-yl}-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-{6-[(4-pyrimidin-2-ylpiperazin-1-yl)methyl]pyridin-3-yl}pyrimidin-2-amine;
5-Fluoro-N-(6-{[(2S)-2-(methoxymethyl)pyrrolidin-1-yl]methyl}pyridin-3-yl)-4-[2-methyl-1-(tetrahydro-2H-pyran-4-yl)-1H-imidazol-5-yl]pyrimidin-2-amine;
N-{6-[(4-Acetyl-1,4-diazepan-1-yl)methyl]pyridin-3-yl}-5-fluoro-4-[2-methyl-1-(tetrahydro-2H-pyran-4-yl)-1H-imidazol-5-yl]pyrimidin-2-amine;
N-{6-[(2,6-Dimethylmorpholin-4-yl)methyl]pyridin-3-yl}-5-fluoro-4-[2-methyl-1-(tetrahydro-2H-pyran-4-yl)-1H-imidazol-5-yl]pyrimidin-2-amine;
N-{6-[(4,4-Difluoropiperidin-1-yl)methyl]pyridin-3-yl}-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[6-(pyrrolidin-1-ylmethyl)pyridin-3-yl]pyrimidin-2-amine;
N-[6-({[(6-Chloropyridin-3-yl)methyl]amino}methyl)pyridin-3-yl]-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[6-(1,4-oxazepan-4-ylmethyl)pyridin-3-yl]pyrimidin-2-amine;
5-Fluoro-N-{6-[(4-methoxypiperidin-1-yl)methyl]pyridin-3-yl}-4-[2-methyl-1-(tetrahydro-2H-pyran-4-yl)-1H-imidazol-5-yl]pyrimidin-2-amine;
(1-{[5-({5-Fluoro-4-[2-methyl-1-(tetrahydro-2H-pyran-4-yl)-1H-imidazol-5-yl]pyrimidin-2-yl}amino)pyridin-2-yl]methyl}piperidin-3-yl)methanol;
1-[3-({[5-({5-Fluoro-4-[2-methyl-1-(tetrahydro-2H-pyran-4-yl)-1H-imidazol-5-yl]pyrimidin-2-yl}amino)pyridin-2-yl]methyl}amino)propyl]pyrrolidin-2-one;
5-Fluoro-4-[2-methyl-1-(tetrahydro-2H-pyran-4-yl)-1H-imidazol-5-yl]-N-{6-[(4-pyrrolidin-1-ylpiperidin-1-yl)methyl]pyridin-3-yl}pyrimidin-2-amine;
3-[{[5-({5-Fluoro-4-[2-methyl-1-(tetrahydro-2H-pyran-4-yl)-1H-imidazol-5-yl]pyrimidin-2-yl}amino)pyridin-2-yl]methyl}(tetrahydrofuran-2-ylmethyl)amino]propanenitrile;
N-[6-(Azetidin-1-ylmethyl)pyridin-3-yl]-5-fluoro-4-[2-methyl-1-(tetrahydro-2H-pyran-4-yl)-1H-imidazol-5-yl]pyrimidin-2-amine;
N-(6-{[Ethyl(2-methoxyethyl)amino]methyl}pyridin-3-yl)-5-fluoro-4-[2-methyl-1-(tetrahydro-2H-pyran-4-yl)-1H-imidazol-5-yl]pyrimidin-2-amine;
({[5-({5-Fluoro-4-[2-methyl-1-(tetrahydro-2H-pyran-4-yl)-1H-imidazol-5-yl]pyrimidin-2-yl}amino)pyridin-2-yl]methyl}amino)acetonitrile;
{5-Fluoro-4-[2-methyl-3-(tetrahydro-pyran-4-yl)-3H-imidazol-4-yl]-pyrimidin-2-yl}-isoquinolin-4-yl-amine;
{5-Fluoro-4-[2-methyl-3-(tetrahydro-pyran-4-yl)-3H-imidazol-4-yl]-pyrimidin-2-yl}-pyridin-4-yl-amine;
tert-Butyl 4-({5-fluoro-4-[2-methyl-1-(tetrahydro-2H-pyran-4-yl)-1H-imidazol-5-yl]pyrimidin-2-yl}amino)piperidine-1-carboxylate;
5-Fluoro-4-[2-methyl-1-(tetrahydro-2H-pyran-4-yl)-1H-imidazol-5-yl]-N-(tetrahydro-2H-pyran-4-yl)pyrimidin-2-amine;
N-(1-Acetylpiperidin-4-yl)-5-fluoro-4-[2-methyl-1-(tetrahydro-2H-pyran-4-yl)-1H-imidazol-5-yl]pyrimidin-2-amine;
N-Cyclohexyl-5-fluoro-4-[2-methyl-1-(tetrahydro-2H-pyran-4-yl)-1H-imidazol-5-yl]pyrimidin-2-amine;
N-(1-Benzylpiperidin-4-yl)-5-fluoro-4-[2-methyl-1-(tetrahydro-2H-pyran-4-yl)-1H-imidazol-5-yl]pyrimidin-2-amine;
N-(1-Benzoylpiperidin-4-yl)-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[1-(phenylacetyl)piperidin-4-yl]pyrimidin-2-amine;
Benzyl 4-({5-fluoro-4-[2-methyl-1-(tetrahydro-2H-pyran-4-yl)-1H-imidazol-5-yl]pyrimidin-2-yl}amino)piperidine-1-carboxylate;
5-Fluoro-N-[1-(methylsulfonyl)piperidin-4-yl]-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[1-(phenylsulfonyl)piperidin-4-yl]pyrimidin-2-amine;
N-[1-(Benzylsulfonyl)piperidin-4-yl]-5-fluoro-4-[2-methyl-1-(tetrahydro-2H-pyran-4-yl)-1H-imidazol-5-yl]pyrimidin-2-amine; and
5-Fluoro-4-[2-methyl-1-(tetrahydro-2H-pyran-4-yl)-1H-imidazol-5-yl]-N-[1-(trifluoroacetyl)piperidin-4-yl]pyrimidin-2-amine;
   as a free base or a pharmaceutically acceptable salt thereof.

The present invention also provides a compound selected from:
5-({5-Fluoro-4-[2-methyl-1-(tetrahydro-2H-pyran-4-yl)-1H-imidazol-5-yl]pyrimidin-2-yl}amino)pyridine-2-carbaldehyde; and
2-Bromo-5-fluoro-4-[2-methyl-1-(tetrahydro-2H-pyran-4-yl)-1H-imidazol-5-yl]pyrimidine. Said compound(s) can be used as intermediates in processes for obtaining a compound of formula (I).

In this specification the term “alkyl” includes both straight and branched chain alkyl groups but references to individual alkyl groups such as “propyl” are specific for the straight chain version only. For example, “C₁₋₆alkyl” and “C₁₋₄alkyl” include methyl, ethyl, propyl, isopropyl and t-butyl. Also, for example “C₆alkyl” is intended to include straight and branched chain alkyl groups having 6 carbon atoms, such as hexan-1-yl, hexan-2-yl and hexan-3-yl. However, references to individual alkyl groups such as ‘propyl’ are specific for the straight-chained version only and references to individual branched chain alkyl groups such as ‘isopropyl’ are specific for the branched chain version only. A similar convention applies to other radicals, for example “carbocyclylC₁₋₆alkyl-R²² includes carbocyclylmethyl-R²², 1-carbocyclylethyl-R²² and 2-carbocyclylethyl-R²².

In this specification the term “alkenyl” includes both straight and branched chain alkenyl groups. For example, “C₂₋₆alkenyl” and “C₂₋₄alkenyl” include allyl, ethenyl, 2-methylprop-1-enyl, but-1-enyl, but-2-enyl and 2-methylbut-2-enyl. Also, for example “C₆alkenyl” is intended to include straight and branched chain alkenyl groups having 6 carbon atoms, such as hex-4-enyl, hex-5-enyl and 2-methyl-pent-3-enyl

In this specification the term “alkynyl” includes both straight and branched chain alkynyl groups. For example, “C₂₋₆alkynyl” include ethynyl, propynyl, but2-ynyl and 2-methylpent-2-ynyl. Also, for example “C₆alkynyl” is intended to include straight and branched chain alkynyl groups having 6 carbon atoms such as 2-methylpent-2-ynyl and hex-4-ynyl.

The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” or “heterocycle” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides.

Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” or “carbocycle” is a saturated, partially saturated or unsaturated, mono or bicyclic carbon ring that contains 3-12 atoms; wherein a CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “N—(C₁₋₆alkyl)sulphamoyl” are N-(methyl)sulphamoyl and N-(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “C₁₋₆alkylsulphonylamino” include methylsulphonylamino, isopropylsulphonylamino and t-butylsulphonylamino. Examples of “C₁₋₆alkylsulphonyl” include methylsulphonyl, isopropylsulphonyl and t-butylsulphonyl.

The terms “—C₁₋₄alkylcarbocyclyl” and “—C₁₋₄alkylheterocyclyl” include both straight and branched chain alkyl groups of between one and four carbon atoms that then link to a carbocycle or heterocycle respectively. The terms carbocycle and heterocycle are as defined above. Non-limiting examples of —C₁₋₄alkylcarbocyclyl therefore include benzyl, 2-phenylethyl, 1-phenylethyl, cyclopropylmethyl and cyclohexylethyl. Non-limiting examples of —C₁₋₄alkylheterocyclyl include pyridin-3-ylmethyl, oxolan-2-yl-methyl, 2-(4-piperidyl)ethyl and 1-thiophen-2-ylethyl.

The terms “—C₁₋₃alkylhydroxy”, “—C₁₋₃alkylmethoxy”, “—C₁₋₃alkylethoxy” and “—C₁₋₃alkylisopropoxy” include both straight and branched chain alkyl groups of between one and three carbon atoms that then link to a hydroxy, methoxy, ethoxy or isopropoxy group respectively. Non-limiting examples of “—C₁₋₃alkylhydroxy” include hydroxymethyl, 1-hydroxyethyl and 2-hydroxyethyl. Non-limiting examples of “—C₁₋₃alkylmethoxy” include methoxymethyl, 1-methoxyethyl and 2-methoxyethyl. Non-limiting examples of “—C₁₋₃alkylethoxy” include ethoxymethyl, 1-ethoxyethyl and 2-ethoxyethyl. Non-limiting examples of “—C₁₋₃alkylisopropoxy” include isopropoxymethyl, 1-isopropoxyethyl and 2-isopropoxyethyl.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

An in vivo hydrolysable ester of a compound of formula (I) containing carboxy or hydroxy group is, for example, a pharmaceutically acceptable ester that is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

Some compounds of the formula (I) may have stereogenic centres and/or geometric isomeric centres (E- and Z-isomers), and it is to be understood that the invention encompasses all such optical isomers, diastereoisomers and geometric isomers that possess GSK3 inhibitory activity.

The invention relates to any and all tautomeric forms of the compounds of the formula (I) that possess GSK3 inhibitory activity.

The definition of compounds of formula (I) also includes in vivo hydrolysable esters, solvates or solvates of salts thereof.

It is also to be understood that certain compounds of the formula (I) can exist in solvated as well as unsolvated forms such as, for example, hydrated forms. It is to be understood that the invention encompasses all such solvated forms that possess GSK3 inhibitory activity.

### Methods of Preparation

The present invention also provides a process for preparing a compound of formula (I), or a pharmaceutically acceptable salt thereof, or an in vivo hydrolysable ester thereof, which process comprises the following steps:

a) reacting a pyrimidine of formula (II):

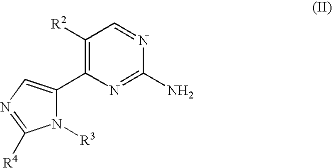

with a compound of formula (III):

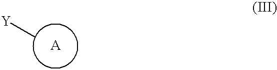

wherein R¹, R², R³, R⁴ and A are, unless otherwise specified, as defined in formula (I);
wherein A contains an aromatic mono- or bicyclic heterocycle;
wherein Y is a displaceable group;
and thereafter optionally:
b) converting a compound of formula (I) into another compound of formula (I);
c) removing any protecting groups; and
d) forming a pharmaceutically acceptable salt or in vivo hydrolysable ester.

Y is a displaceable group, such as a halo or sulphonyloxy group, for example a chloro, bromo, iodo or trifluoromethanesulphonyloxy group. According to one embodiment of the present invention Y is chloro, bromo or iodo.

Specific reaction conditions for the above reactions are as follows:

Step a). Amines of formula (II) and compounds of formula (III) or (IV) may be reacted together under standard Buchwald-Hartwig conditions (for example see J. Am. Chem. Soc., 118, 7215; J. Am. Chem. Soc., 119, 8451; J. Am. Chem. Soc., 125, 6653; J. Org. Chem., 62, 1568 and 6066) for example in the presence of palladium acetate, in a suitable solvent for example an aromatic solvent such as toluene, benzene or xylene, with a suitable base for example an inorganic base such as caesium carbonate or an organic base such as potassium-t-butoxide, in the presence of a suitable ligand such as 2,2′-bis(diphenylphosphino)-1,1′-binaphthyl or 2-dicyclohexylphosphino-2′,4′,6′-triiso-propyl-1,1′-biphenyl and at a temperature in the range of +25 to +90° C.

Pyrimidines of the formula (II), wherein R³ is methyl and R⁴ and R² are defined as in formula (I), may be prepared according to Scheme 1:

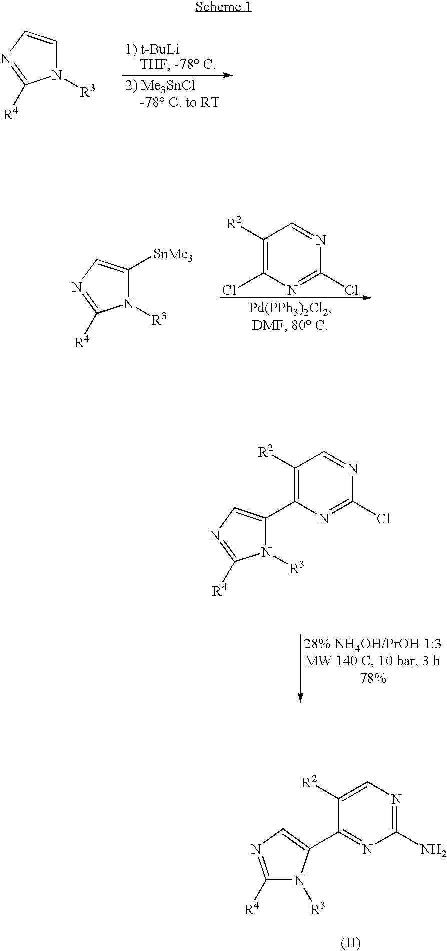

An alternative synthesis of pyrimidines of formula (II) is described in Scheme 2 (wherein R^{x} is selected from the same or different C₁₋₆alkyl, and R², R³ and R⁴ are as defined in formula (I)):

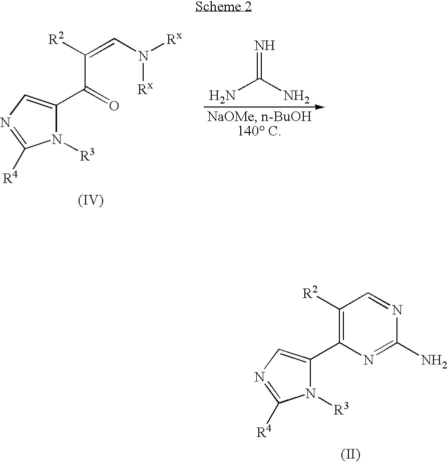

Compounds of formula (III) are commercially available compounds, or they are known in the literature, or they can be prepared by standard processes known in the art.

Compounds of formula (IV) in which R³ has the general structure R^{a}—CH—R^{b}, wherein R^{a} and R^{b} are hydrogen or form together a tetrahydropyran ring, wherein R⁴ is hydrogen or C₁₋₃alkyl, wherein said C₁₋₃alkyl may optionally be substituted with one or more halo and wherein R² is fluoro and R^{x} is as defined above may be prepared according to Scheme 3:

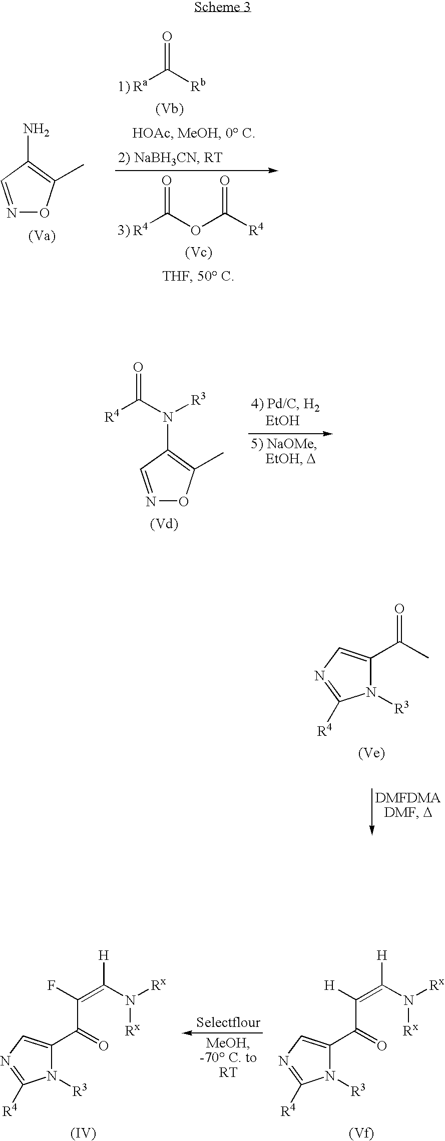

Compounds of formula (Va), (Vb) and (Vc) are commercially available compounds, or they are known in the literature, or they can be prepared by standard processes known in the art. The compound of formula (Vf) can exist in both E and Z forms.

Furthermore, compounds of formula (Ia) can also be prepared by the reaction of an intermediate such as compound VI, which is prepared from a compound of formula (II) by reaction with TMSBr and tert-butylnitrite in a polar aprotic solvent, wherein R¹, R², R³, R⁴ and A are, unless otherwise specified, as defined in formula (I); A is a saturated or partially saturated carbocycle or a saturated or partially saturated heterocycle.

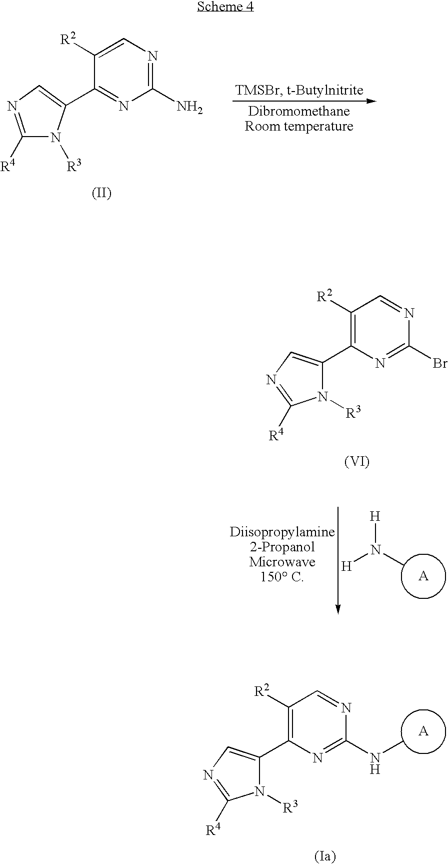

A can also be a protected saturated or partially saturated heterocycle (e.g. tert-butoxycarbonyl protected piperidine) or a saturated or partially saturated carbocycle with a protected substituent (e.g. tert-butoxycarbonyl protected amino, substituted on a cyclohexyl ring) and in such cases further compounds of formula (Ia) can be prepared by removing the protecting group and then reacting the amine in order to obtain, for example, amides or sulphonamides. This is shown in Scheme 5, in which the starting compound of formula (Ib) (wherein R³ is 4-tetrahydropyranyl, R⁴ is methyl, R² is fluoro, A is 4-piperidinyl, R⁵ is —C(═O)O— and R⁷ is tert-butyl) is deprotected to give a secondary amine, said amine is reacted to give either a compound of formula (Id) (wherein R⁵ is C(O) and R⁷ is as defined above), or a compound of formula (Ic) (wherein R⁵ is SO₂ and R⁷ is as defined above). The deprotection of the compound of formula (Ib) can be performed in acidic media or solvents such as trifluoroacetic acid (TFA) or anhydrous hydrochloric acid in methanol. The amide couplings to obtain compounds of formula (Id) can be performed using standard amide coupling reagents in a polar, aprotic solvent in the presence of a base.

The sulphonamides of formula (Ic) can be prepared by reaction with sulphonyl halides (such as fluoro, chloro or bromo) in a polar aprotic solvent in the presence of a base.

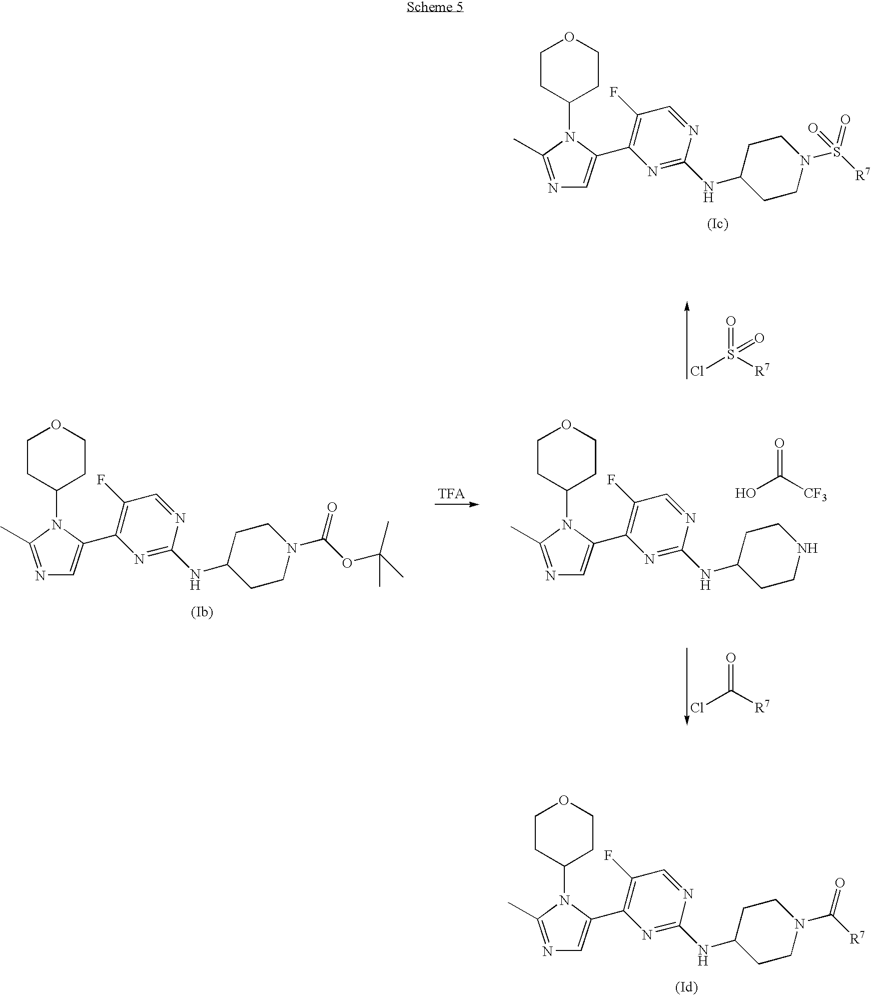

A compound of formula (Ie) can be prepared by reacting an aldehyde intermediate of formula (VII) reductively with primary or secondary amines as shown in Scheme 6. This reaction can be achieved by mixing said aldehyde with an amine in a polar, aprotic solvent to form an imine, this is then followed by the reduction of the imine to an amine. The reductive amination conditions involve, for example, having a mixture of the amine and aldehyde in NMP and adding to said mixture, after imine formation, sodium cyanoborohydride or sodium triacetoxyborohydride.

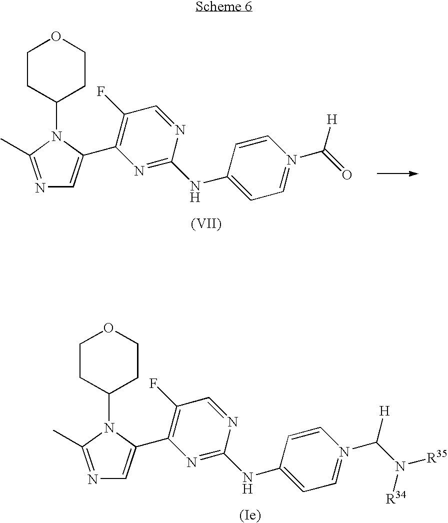

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Greene, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group that may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

### General Methods

All solvents used were analytical grade and commercially available anhydrous solvents were routinely used for reactions. Reactions were typically run under an inert atmosphere of nitrogen or argon.

¹H, ¹⁹F and ¹³C NMR spectra were recorded on a Varian Unity+400 NMR Spectrometer equipped with a 5 mm BBO probehead with Z-gradients, or a Varian Gemini 300 NMR spectrometer equipped with a 5 mm BBI probehead, or a Bruker Avance 400 NMR spectrometer equipped with a 60 μl dual inverse flow probehead with Z-gradients, or a Bruker DPX400 NMR spectrometer equipped with a 4-nucleus probehead equipped with Z-gradients, or a Bruker Avance 600 NMR spectrometer equipped with a 5 mm BBI probehead with Z-gradients. Unless specifically noted in the examples, spectra were recorded at 400 MHz for proton, 376 MHz for fluorine-19 and 100 MHz for carbon-13. The following reference signals were used: the middle line of DMSO-d₆ δ 2.50 (1H), δ 39.51 (13C); the middle line of CD₃OD δ 3.31 (1H) or δ 49.15 (13C); CDCl₃ δ 7.26 (1H) and the middle line of CDCl₃ δ 77.16 (13C) (unless otherwise indicated). NMR spectra are either reported from high to low field or from low to high field.

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A: 95:5 10 mM NH₄OAc:MeCN, or 95:5 8 mM HCOOH:MeCN) ending at 100% B (MeCN).

Alternatively, mass spectra were recorded on a Waters LCMS consisting of an Alliance 2690 Separations Module, Waters 2487 Dual 1 Absorbance Detector (220 and 254 nm) and a Waters ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 97-800 with a scan time of 0.3 or 0.8 s. Separations were performed on a Chromolith Performance RP-18e (100×4.6 mm). A linear gradient was applied starting at 95% A (A: 0.1% HCOOH (aq.)) ending at 100% B (MeCN) in 5 minutes. Flow rate: 2.0 mL/min.

Microwave heating was performed in a single-mode microwave cavity producing continuous irradiation at 2450 MHz.

HPLC analyses were performed on an Agilent HP 1000 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (A: 95:5 10 mM NH₄OAc:MeCN) and ending at 100% B (B: MeCN), in 4 min.

Alternatively, HPLC analyses were performed on a Gynkotek P580 HPG consisting of gradient pump with a Gynkotek UVD 170S UV-vis.-detector equipped with a Chromolith Performance RP column (C18, 100 mm×4.6 mm). The column temperature was set to 25° C. A linear gradient was applied using MeCN/0.1 trifluoroacetic acid in MilliQ water, run from 10% to 100% MeCN in 5 minutes. Flow rate: 3 ml/min.

A typical workup procedure after a reaction consisted of extraction of the product with a solvent such as ethyl acetate, washing with water followed by drying of the organic phase over MgSO₄ or Na₂SO₄, filtration and concentration of the solution in vacuo.

Thin layer chromatography (TLC) was performed on Merck TLC-plates (Silica gel 60 F₂₅₄) and UV visualized the spots. Flash chromatography was performed on a Combi Flash® Companion™ using RediSep™ normal-phase flash columns or using Merck Silica gel 60 (0.040-0.063 mm). Typical solvents used for flash chromatography were mixtures of chloroform/methanol, dichloromethane/methanol, heptane/ethyl acetate, chloroform/methanol/ammonia (aq.) and dichloromethane/methanol/NH₃ (aq.). SCX ion exchange columns were performed on Isolute® columns. Chromatography through ion exchange columns were typically performed in solvents such a methanol.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 ml/min.

The formation of hydrochloride salts of the final products were typically performed in solvents or solvents mixtures such as diethyl ether, tetrahydrofuran, dichloromethane/toluene, dichloromethane/methanol, followed by addition of 1M hydrogen chloride in diethyl ether.

The following abbreviations have been used:

aq. aqueous;
Ar (g) Argon gas;
CDCl₃ deuterated chloroform;
CHCl₃ chloroform;
CH₂Cl₂ dimethylchloride;
Cs₂CO₃ caesium carbonate;

### DMF N,N-dimethylformamide;

DMFDMA dimethylformamide dimethylacetal;
DMSO dimethyl sulphoxide;
DMSO-d₆ deuterated dimethyl sulphoxide;
EtOAc ethyl acetate;
EtOH ethanol;
HCOOH acetic acid;
HCl hydrochloride;
HOAc acetic acid;
MeCN acetonitrile;
MeOH methanol;
MeOD deuterated methanol;
Me₃SnCl trimethyltin chloride;
MgSO₄ magnesium sulphate;
Min minutes;
NaBH(OAc)₃ sodium triacetoxyborohydride;
NaHCO₃ sodium bicarbonate;
NaOMe sodium methoxide;
Na₂SO₄ sodium sulphate;
n-BuOH n-butanol;
NH₃ ammonia;
NH₄OAc ammonium acetate;
NH₄OH ammonium hydroxide;
Pd/C palladium on carbon;
Pd(PPh₃)₂Cl₂ bis(triphenylphosphine)palladium dichloride;
Pd₂(dba)₃ tris(dibenzylideneacetone)dipalladium;
PrOH propan-1-ol;
r.t. or RT room temperature;
Ret. T retention time
Selectfluor N-fluoro-N′-chloromethyl-triethylenediamine-bis(tetrafluoroborate);
t-BuLi tert-butyllithium;
THF tetrahydrofuran;
TMSBr trimethylsilylbromide;
Xantphos 9,9-Dimethyl-4,5-bis(diphenylphosphino)xanthene;
X-Phos 2-dicyclohexylphosphino-2′,4′,6′-triiso-propyl-1,1′-biphenyl.

Starting materials used were either available from commercial sources or prepared according to literature procedures and had experimental data in accordance with those reported.

Compounds have been named either using ACD/Name, version 9, software from Advanced Chemistry Development, Inc. (ACD/Labs), Toronto ON, Canada, www.acdlabs.com, 2004 or named according to the IUPAC convention.

### General Methods A to D

In the following general methods A to D, the groups R¹, R², R³, R⁴, halo and A are used independently to indicate the diversity of substitution within each structure. The identity of R¹, R², R³, R⁴, halo and A will be clear to a person skilled in the art based on the starting materials and intermediates for each specific example. For instance in Example 1, which refers to General method A, A1 is 5-fluoro-4-[2-methyl-1-(tetrahydro-2H-pyran-4-yl)-1H-imidazol-5-yl]pyrimidin-2-amine such that R³ is 4-tetrahydropyranyl and A2 is 5-bromopyrimidine such that A is pyrimidine and Halo is bromo- at the 5-position of the pyrimidine ring.

### General Method A

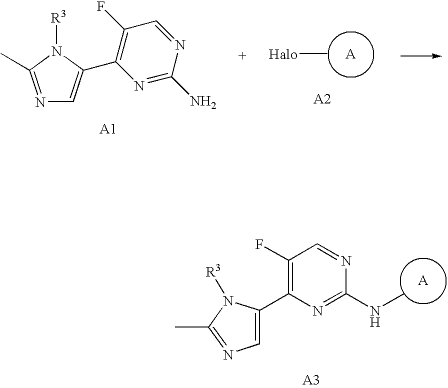

A1 (1.01-1.27 equiv.), A2 (1.0 equiv.) (wherein A and R³ are as defined in formula (I) and Halo is Cl, Br or I) and Cs₂CO₃ (1.66-2.25 equiv.) were mixed in anhydrous 1,4-dioxane and the mixture was flushed with argon for 5 minutes before Pd₂(dba)₃ (0.05-0.08 equiv.) and X-Phos or Xantphos (0.10-0.16 equiv.) were added. The mixture was flushed with argon, then heated in a sealed tube at +90-+100° C. until the reaction was complete. The solvent was removed in vacuo and the residue was taken up in CH₂Cl₂ and washed with diluted NaHCO₃ (aq.) or water. The organic layer was dried (Na₂SO₄), filtered and evaporated. The crude of the base product was purified using preparative HPLC. Optionally, the mono- or di-hydrochloride salt was made by dissolving the compound in a solvent such as diethyl ether, tetrahydrofuran, dichloromethane, dichloromethane/toluene or dichloromethane/methanol, followed by addition of 1M hydrogen chloride in diethyl ether.

### General Method B

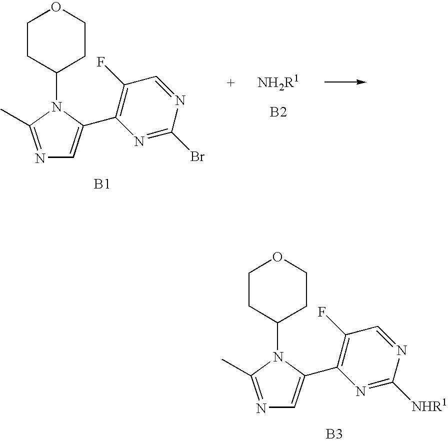

2-Bromo-5-fluoro-4-[2-methyl-1-(tetrahydro-2H-pyran-4-yl)-1H-imidazol-5-yl]pyrimidine (B1) (1.0 equiv.), a primary amine B2 (2.0 equiv), diisopropylethylamine (2.0-5.0 equiv) and 2-propanol was added to a microwave tube and heated for 6 hours at 150° C. in the micro wave. The solvent was evaporated in vacuo and the crude product was purified using preparative HPLC.

### General Method C

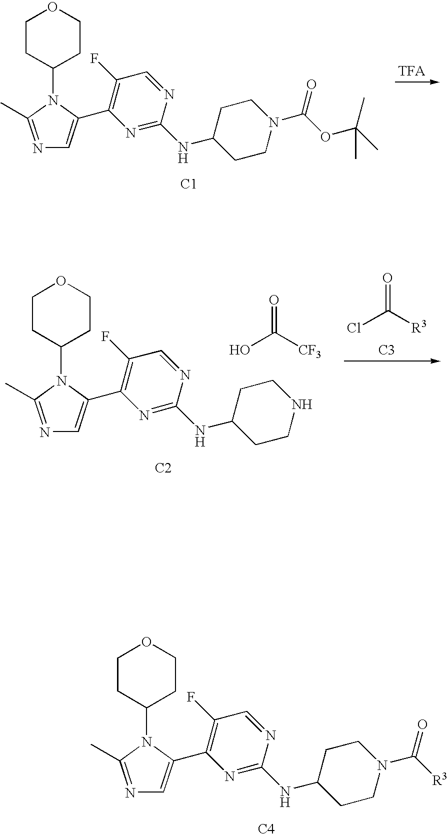

tert-Butyl 4-({5-fluoro-4-[2-methyl-1-(tetrahydro-2H-pyran-4-yl)-1H-imidazol-5-yl]pyrimidin-2-yl}amino)piperidine-1-carboxylate (C1) (1.0 equiv.) was dissolved in TFA and stirred at r.t. for 30 minutes. The solvent was evaporated in vacuo and the residue (C2) was dissolved in CH₂Cl₂. Diisopropylethylamine (2.5 equiv) was added, followed by the acid chloride C3 (1 equiv.) and the mixture was stirred at r.t. for 1 hour before it was diluted with CH₂Cl₂, washed with saturated NaHCO₃ (aq.), dried (Na₂SO₄) and filtered. The solvent was evaporated in vacuo and the crude product was purified using preparative HPLC.

### General Method D

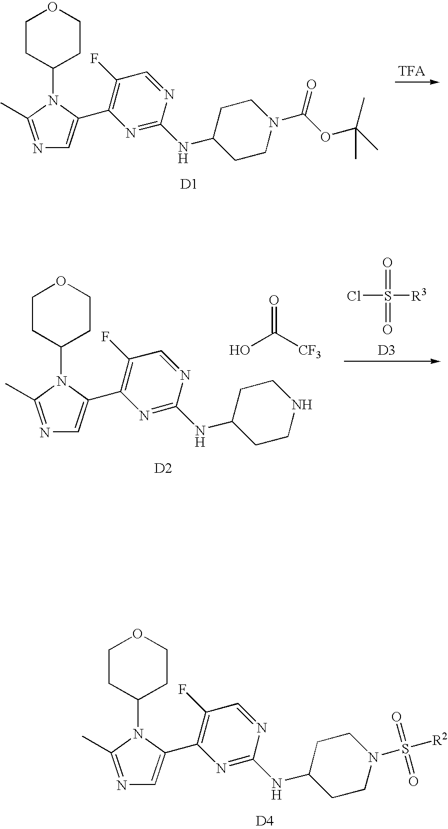

tert-Butyl 4-({5-fluoro-4-[2-methyl-1-(tetrahydro-2H-pyran-4-yl)-1H-imidazol-5-yl]pyrimidin-2-yl}amino)piperidine-1-carboxylate, D1, (1.0 equiv.) was dissolved in TFA and stirred at r.t. for 30 minutes. The solvent was evaporated in vacuo and the residue (D2) was dissolved in CH₂Cl₂. Diisopropylethylamine (2.5 equiv.) was added followed by sulphonyl chloride D3 (1 equiv.) and the mixture was stirred at r.t. for 1 hour before it was diluted with CH₂Cl₂, washed with saturated NaHCO₃ (aq.), dried (Na₂SO₄) and filtered. The solvent was evaporated in vacuo and the crude product was purified using preparative HPLC.

### EXAMPLES

The present invention will further be described in more detail by the following Examples, which are not to be construed as limiting the present invention.

### Example 1

### 5-Fluoro-4-[2-methyl-1-(tetrahydro-2H-pyran-4-yl)-1H-imidazol-5-yl]-N-pyrimidin-5-ylpyrimidin-2-amine

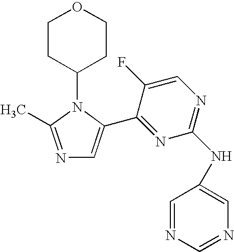

The title compound was prepared in accordance with the general method A using 5-fluoro-4-[2-methyl-1-(tetrahydro-2H-pyran-4-yl)-1H-imidazol-5-yl]pyrimidin-2-amine (50 mg, 0.18 mmol) and 5-bromopyrimidine (29 mg, 0.18 mmol) to give the title compound (13 mg, 20%).

¹H NMR (CDCl₃) δ ppm 9.04 (s, 2H) 8.92 (s, 1H) 8.36 (d, J=2.78 Hz, 1H) 7.68 (s, 1H) 7.44 (s, 1H) 5.02-5.12 (m, 1H) 4.09 (d, J=4.55 Hz, 1H) 4.06 (d, J=4.80 Hz, 1H) 3.26 (td, J=11.87, 1.77 Hz, 2H) 2.65 (s, 3H) 2.44-2.55 (m, 2H) 1.86 (dd, J=2.78, 3.03 Hz, 2H); MS (ES) m/z 356 (M+1).

### Example 2

### 1-[5-({5-Fluoro-4-[2-methyl-1-(tetrahydro-2H-pyran-4-yl)-1H-imidazol-5-yl]pyrimidin-2-yl}amino)pyridin-3-yl]ethanone

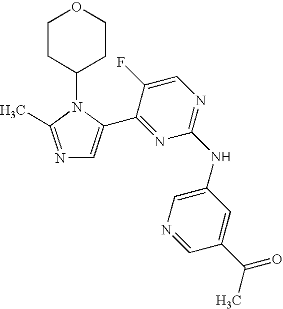

The title compound was prepared in accordance with the general method A using 5-fluoro-4-[2-methyl-1-(tetrahydro-2H-pyran-4-yl)-1H-imidazol-5-yl]pyrimidin-2-amine (50 mg, 0.18 mmol) and 1-(5-bromopyridin-3-yl)ethanone (34 mg, 0.17 mmol) to give the title compound (29 mg, 43%) which was later transformed to the hydrochloride salt as described in general method A.

¹H NMR (HCl salt, DMSO-d) δ ppm 10.46 (s, 1H) 9.20 (s, 1H) 8.91 (m, 2H) 8.62 (s, 1H) 8.16 (s, 1H) 4.97 (m, 1H) 3.82 (m, 2H) 3.20 (m, 2H) 2.85 (s, 3H) 2.65 (s, 3H) 2.16 (m, 2H) 1.94 (m, 2H); MS (ES) m/z 397 (M+1).

### Example 3

### 5-Fluoro-N-(6-methoxypyridin-2-yl)-4-[2-methyl-1-(tetrahydro-2H-pyran-4-yl)-1H-imidazol-5-yl]pyrimidin-2-amine

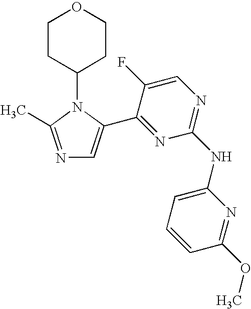

The title compound was prepared in accordance with general method A using 5-fluoro-4-[2-methyl-1-(tetrahydro-2H-pyran-4-yl)-1H-imidazol-5-yl]pyrimidin-2-amine (35 mg, 0.13 mmol) and 2-bromo-6-methoxypyridine (21 mg, 0.11 mmol) to give the title compound (38 mg, 87%) which was later transformed to the hydrochloride salt as described in general method A.

¹H NMR (HCl salt, DMSO-d) δ ppm 10.06 (s, 1H), 8.87 (s, 1H), 8.13 (s, 1H), 7.60 (m, 2H), 4.45 (m, 1H), 5.03 (m, 1H), 3.84 (s, 3H), 3.25 (m, 2H), 2.85 (s, 3H), 2.17 (m, 2H), 1.97 (m, 2H); MS (ES) m/z 385 (M+1).

### Example 4

### 5-Fluoro-4-[2-methyl-1-(tetrahydro-2H-pyran-4-yl)-1H-imidazol-5-yl]-N-[5-(trifluoromethyl)pyridin-2-yl]pyrimidin-2-amine

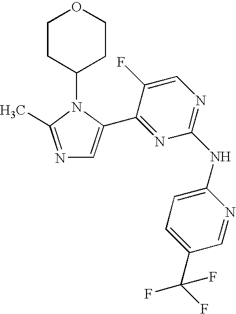

The title compound was prepared in accordance with general method A using 5-fluoro-4-[2-methyl-1-(tetrahydro-2H-pyran-4-yl)-1H-imidazol-5-yl]pyrimidin-2-amine (35 mg, 0.13 mmol) and 2-chloro-5-(trifluoromethyl)pyridine (21 mg, 0.11 mmol) to give the title compound (40 mg, 84%) which was later transformed to the hydrochloride salt as described in general method A.

¹H NMR (HCl salt, DMSO-d) δ ppm 10.95 (s, 1H), 8.95 (s, 1H), 8.68 (s, 1H), 8.16 (m, 3H), 5.05 (m, 1H), 3.86 (m, 2H), 3.28 (m, 2H), 2.85 (s, 3H), 2.20 (m, 2H), 2.00 (m, 2H); MS (ES) m/z 423 (M+1).

### Example 5

### 5-Fluoro-N-(6-methylpyridin-3-yl)-4-[2-methyl-1-(tetrahydro-2H-pyran-4-yl)-1H-imidazol-5-yl]pyrimidin-2-amine

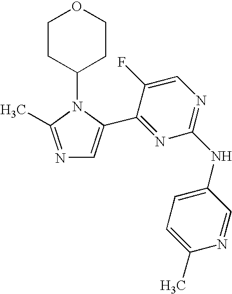

The title compound was prepared in accordance with general method A using 5-fluoro-4-[2-methyl-1-(tetrahydro-2H-pyran-4-yl)-1H-imidazol-5-yl]pyrimidin-2-amine (35 mg, 0.13 mmol) and 2-bromo-5-methylpyridine (22 mg, 0.13 mmol) to give the title compound (22 mg, 48%) which was later transformed to the hydrochloride salt as described in general method A.

¹H NMR (HCl salt, DMSO-d) δ ppm 10.68 (s, 1H), 9.02 (s, 1H), 8.95 (s, 1H), 8.54 (m, 1H), 8.14 (s, 1H), 7.78 (m, 1H), 4.96 (m, 1H), 3.87 (m, 2H), 3.27 (m, 2H), 2.85 (s, 3H), 2.67 (s, 3H), 2.17 (m, 2H), 1.97 (m, 2H); MS (ES) m/z 369 (M+1).

### Example 6

### 5-Fluoro-N-(4-methoxypyridin-2-yl)-4-[2-methyl-1-(tetrahydro-2H-pyran-4-yl)-1H-imidazol-5-yl]pyrimidin-2-amine

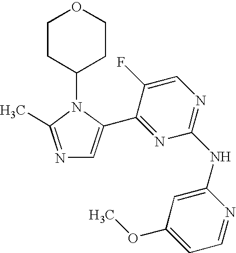

The title compound was prepared in accordance with general method A using 5-fluoro-4-[2-methyl-1-(tetrahydro-2H-pyran-4-yl)-1H-imidazol-5-yl]pyrimidin-2-amine (40 mg, 0.14 mmol) and 2-chloro-4-methoxypyridine (23 mg, 0.16 mmol) to give the title compound (49 mg, 88%) which was later transformed to the hydrochloride salt as described in general method A.

¹H NMR (HCl salt, DMSO-d) δ ppm 12.21 (s, 1H), 9.01 (s, 1H), 8.34 (m, 1H), 8.22 (s, 1H), 7.65 (s, 1H), 7.05 (m, 1H), 5.05 (m, 1H), 4.01 (s, 3H), 3.91 (m, 2H), 3.41 (m, 2H), 2.87 (s, 3H), 2.20 (m, 2H), 2.04 (m, 2H); MS (ES) m/z 385 (M+1).

The main intermediates were prepared as followed:

### Example 7

### 5-Fluoro-4-[2-methyl-1-(tetrahydro-2H-pyran-4-yl)-1H-imidazol-5-yl]pyrimidin-2-amine

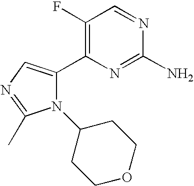

### Example 7(a) 4-[N-Acetyl-N-(tetrahydro-2H-pyran-4-yl)]amino-5-methylisoxazole

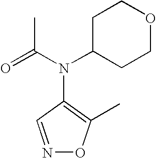

5-Methyl-4-amino-isoxazole (Reiter, L. A., J. Org. Chem. 1987, 52, 2714-2726) (0.68 g, 5.1 mmol) and acetic acid (0.61 g, 10.2 mmol) were dissolved in MeOH (20 mL). Tetrahydro-2H-pyran-4-one (0.76 g, 7.6 mmol) was added and the mixture was cooled to 0-(−5)° C. and stirred for 1 h. Sodium cyanoborohydride (0.32 g, 5.1 mmol) was added to the reaction mixture at −5° C., causing weak exothermic and gas evolution. The cooling bath was removed and the mixture was stirred at r.t. for 1 h, followed by the addition of a second portion of sodium cyanoborohydride (0.1 g, 1.6 mmol). After stirring for 2 h at r.t., the mixture was filtered and the filtrate was concentrated in vacuo. The residue was dissolved in toluene and re-concentrated. The residue was dissolved in THF (10 mL) and acetic anhydride (1.56 g, 15.3 mmol) was added. The resulting mixture was stirred overnight at r.t. then for 1 h at +50° C. The volatiles were removed in vacuo and the residue was dissolved in toluene and concentrated in vacuo to give the title compound (1.36 g, 78%).

¹H NMR (CDCl₃) ppm δ 8.04 (s, 1H), 4.86-4.73 (m, 1H), 4.00-3.89 (m, 2H), 3.52-3.42 (m, 2H), 2.35 (s, 3H), 1.81 (s, 3H), 1.70-1.57 (m, 2H), 1.49-1.23 (m, 2H);

MS (ESI) m/z 225 (M+1).

### Example 7(b) 5-Acetyl-2-methyl-1-(tetrahydro-2H-pyran-4-yl)-1H-imidazole

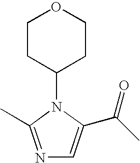

4-[N-Acetyl-N-(tetrahydro-2H-pyran-4-yl)]amino-5-methylisoxazole (4.8 g, 21.4 mmol) was dissolved in EtOH (30 ml), and the mixture was hydrogenated over Pd/C (10%, wet paste, 0.10 g) at 3 bar. The reaction mixture was stirred at 50° C. for 3 h. An additional amount of Pd/C (10%, wet paste, 0.15 g) was added and the mixture was continued stirring at +50° C. for 3 h. Sodium methoxide (1.70 g, 31.46 mmol) was added and the resulting mixture was heated to reflux for 30 h. Ammonium chloride was added to quench the reaction. The mixture was filtrated through diatomaceous earth and the filtrate was evaporated in vacuo. The residue was diluted with saturated sodium bicarbonate (aq.) and extracted with EtOAc, then with CHCl₃. The combined organic layers were dried (Na₂SO₄) and concentrated in vacuo. The crude product was purified by flash chromatography (EtOAc) to give the title compound (3.7 g, 83%).

¹H NMR (CDCl₃) δ 7.70 (s, 1H), 5.40-5.30 (m, 1H), 4.13-4.01 (m, 2H), 3.57-3.44 (m, 2H), 2.57 (s, 3H), 2.44 (s, 3H), 2.43-2.30 (m, 2H), 1.80-1.72 (m, 2H).

### Example 7(c) (2E)-3-Dimethylamino-1-[2-methyl-1-(tetrahydro-2H-pyran-4-yl)-1H-imidazol-5-yl]prop-2-en-1-one

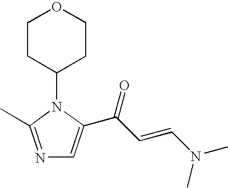

5-Acetyl-2-methyl-1-(tetrahydro-2H-pyran-4-yl)-1H-imidazole (3.7 g, 17.79 mmol) was dissolved in DMFDMA/DMF (1:1, 100 mL) and the mixture was stirred under reflux overnight. After cooling to r.t. the mixture was extracted with CH₂Cl₂. The organic phase was dried (Na₂SO₄), filtered and concentrated in vacuo. The crude product was purified by flash chromatography (CH₂Cl₂/MeOH 15:1) to give the title compound (3.85 g, 82%).

¹H NMR (CDCl₃) δ 7.65 (d, J=12.6 Hz, 1H), 7.46 (s, 1H), 5.55-5.42 (m, 2H), 4.08 (dd, J=11 Hz, 4.4 Hz, 2H), 3.52 (t, J=11 Hz, 2H), 2.99 (br s, 6H), 2.56 (s, 3H), 2.45-2.32 (m, 2H), 1.80-1.72 (m, 2H); MS (ESI) m/z 264 (M+1).

### Example 7(d) (2Z)-3-Dimethylamino-2-fluoro-1-[2-methyl-1-(tetrahydro-2H-pyran-4-yl)-1H-imidazol-5-yl]prop-2-en-1-one

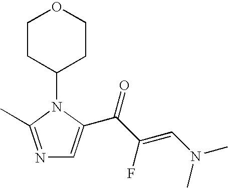

Selectfluor (7.75 g, 21.87 mmol) was added in portions to a stirred solution of (2E)-3-dimethylamino-1-[2-methyl-1-(tetrahydro-2H-pyran-4-yl)-1H-imidazol-5-yl]prop-2-en-1-one (3.85 g, 14.58 mmol) in MeOH (100 mL) at r.t. After stirring at r.t. for 3 h the reaction mixture was cooled in ice/acetone and filtered. The filtrate was evaporated under reduced pressure and the residue was taken into CH₂Cl₂. It was washed with aq. ammonia, brine, dried (Na₂SO₄) and concentrated in vacuo. The crude product was purified by flash chromatography (CH₂Cl₂/MeOH 15:1). The reaction was not run to completion, and the reaction was repeated again with Selectfluor (1.5 equiv.) followed by the same workup. The title compound (1.47 g, 36%).

¹H NMR (CDCl₃, 300 MHz) δ 7.34 (s, 1H), 6.84 (d, J=27.9 Hz, 1H), 5.00-4.88 (m, 1H), 4.04 (dd, J=11.2 Hz, 4.2 Hz, 2H), 3.46 (t, J=11 Hz, 2H), 3.08 (s, 6H), 2.53 (s, 3H), 2.42-2.28 (m, 2H), 1.84-1.75 (m, 2H); MS (ESI) m/z 282 (M⁺+1).

### Example 7(e) 5-Fluoro-4-[2-methyl-1-(tetrahydro-2H-pyran-4-yl)-1H-imidazol-5-yl]pyrimidin-2-amine

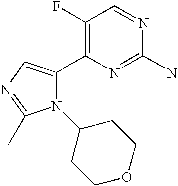

A reaction mixture of (2)-3-dimethylamino-2-fluoro-1-[2-methyl-1-(tetrahydro-2H-pyran-4-yl)-1H-imidazol-5-yl]prop-2-en-one (1.47 g, 5.22 mmol), guanidine carbonate (2.35 g, 13.06 mmol) and sodium methoxide (4.0 equiv.) in 1-butanol was heated in a microwave reactor for 10 minutes at 140° C. under argon or nitrogen atmosphere. The mixture was filtered and the filter was rinsed with CH₂Cl₂. The solvent was evaporated in vacuo and the crude product was purified using flash column chromatography (CH₂Cl₂/MeOH 20:1) to give the title compound (1.21 g, 84%).

¹H NMR (CDCl₃, 300 MHz) δ 8.17 (d, J=3.3 Hz, 1H), 7.59 (d, J=3.9 Hz, 1H), 5.27-5.13 (m, 1H), 4.93 (br s, 2H), 4.13 (dd, J=11.5 Hz, 4.3 Hz, 2H), 3.48 (t, J=11 Hz, 2H), 2.62 (s, 3H), 2.58-2.40 (m, 2H), 1.95-1.84 (m, 2H); MS (ESI) m/z 278 (M+1).

### Example 8

### 4-(1,2-Dimethyl-1H-imidazol-5-yl)-5-fluoropyrimidin-2-amine

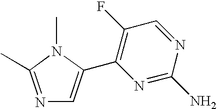

### Example 8(a) 1,2-Dimethyl-5-(trimethylstannyl)-1N-imidazole

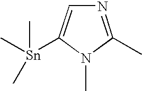

1,2-Dimethylimidazole (0.960 g, 10.0 mmol) was diluted in dry THF (50 mL) under an argon atmosphere and the solution was cooled to −78° C. tert-Butyllithium (1.7M in pentane, 6.47 mL, 11.0 mmol) was added dropwise over 5 minutes. The reaction mixture was stirred for 1 h at −78° C. and then treated with a solution of trimethyltin chloride (2.2 g, 11.0 mmol) in anhydrous THF (10 mL). The mixture was stirred for 60 h from −78° C. to r.t. The solvent was then evaporated in vacuo to give the title compound (1.29 g, 50%). The crude product was used in the next step without further purification.

¹H NMR (CDCl₃) δ ppm 6.87 (s, 1H), 3.56 (s, 3H), 2.41 (s, 3H), 0.45-0.18 (m, 9H); MS (CI) m/z 261 (¹²⁰Sn) (M+1).

### Example 8(b) 2-Chloro-4-(1,2-dimethyl-1H-imidazol-5-yl)-5-fluoropyrimidine

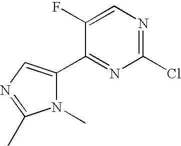

1,2-Dimethyl-5-(trimethylstannyl)-1H-imidazole (0.950 g, 3.68 mmol) and 2,4-dichloro-5-fluoropyrimidine (0.601 g, 3.60 mmol) were diluted in anhydrous DMF (20 mL) and the solution was degassed with argon. Pd(PPh₃)₂Cl₂ (0.126 g, 0.17 mmol) was added and the reaction mixture was stirred at +80° C. for 15 h. The reaction mixture was cooled down to r.t. and concentrated under reduced pressure. Saturated potassium fluoride (aq., 50 mL) is was added and the mixture was stirred for 30 minutes before extraction with EtOAc. The organic layer was dried (MgSO₄), filtered and concentrated under reduced pressure. The crude product was purified by flash chromatography (heptane/EtOAc, 7:3) to give the title compound (0.41 g, 50%).

¹H NMR (CDCl₃, 600 MHz) δ ppm 8.40 (d, J=2.9 Hz, 1H), 7.86 (d, J=4.4 Hz, 1H), 3.97 (s, 3H), 2.53 (s, 3H); MS (ESI) m/z 227 (M+1).

### Example 8(c) 4-(1,2-Dimethyl-1H-imidazol-5-yl)-5-fluoropyrimidin-2-amine

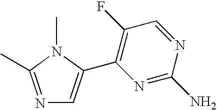

2-Chloro-4-(1,2-dimethyl-1H-imidazol-5-yl)-5-fluoropyrimidine (0.295 g, 1.30 mmol) was dissolved in 1-propanol (3.0 mL) in a microwave vial. Ammonium hydroxide (28%, 1.0 mL) was added, the vial was sealed and the mixture heated in a microwave oven (+140° C., 4 h). The reaction mixture was cooled to r.t. and the solvent was evaporated. The residue was partitioned between CH₂Cl₂ and 1M aqueous HCl. The aqueous phase, containing the product, was neutralized with saturated aqueous NaHCO₃ and the product extracted with CH₂Cl₂. The organic phase was co-evaporated with ethanol and the residue was purified by flash chromatography using (CH₂Cl₂/MeOH gradient; 100:1 to 94:6) to give the title compound (0.210 g, 78%).

¹H NMR (CDCl₃) δ ppm 8.15 (d, J=3.5 Hz, 1H), 7.71 (d, J=4.3 Hz, 1H), 4.87 (br s, 2H), 3.97 (s, 3H), 2.49 (s, 3H); MS (ESI) m/z 208 (M+1).

### Example 9

### 5-Fluoro-4-[1-(tetrahydro-2H-pyran-4-yl)-2-(trifluoromethyl)-1H-imidazol-5-yl]pyrimidin-2-amine

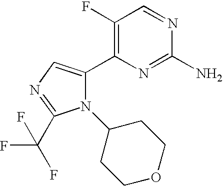

### Example 9(a) 5-Acetyl-1-(tetrahydro-2H-pyran-4-yl)-2-trifluoromethyl-1H-imidazole

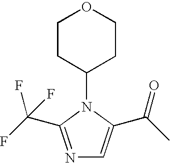

5-Methyl-4-amino-isoxazole (1.7 g, 17.25 mmol) and acetic acid (1.1 g, 19 mmol) were dissolved in methanol (50 mL). Tetrahydro-2H-pyran-4-one (1.9 g, 19 mmol) was added and the mixture was cooled to 0-(−5)° C. and stirred for 1 h. Sodium cyanoborohydride (0.812 g, 12.9 mmol) was added in portions to the reaction mixture at −5° C., causing weak exothermic and gas evolution. The cooling bath was removed and the mixture was stirred at r.t. for 2 h followed by addition of water (20 mL). The methanol was removed from the reaction mixture by vacuum distillation, and the intermediate amine was extracted with ethyl acetate (3×80 mL). The combined organic layers were dried (Na₂SO₄), concentrated to dryness, dissolved in toluene and re-concentrated. The crude intermediate amine, was dissolved in CH₂Cl₂ (20 mL) and pyridine (2 mL, 26 mmol) was added. The mixture was cooled to 0° C. and trifluoroacetic anhydride (4.35 g, 20.7 mmol) was added dropwise. The mixture was continued stirring for 2 h at r.t. and was then washed with water and saturated NaHCO₃. The aqueous layer was extracted with CH₂Cl₂ (2×30 mL), the organic extracts were dried (Na₂SO₄) and concentrated to dryness to give a second crude intermediate, 4-[N-(tetrahydro-2H-pyran-4-yl)]-N-trifluoroacetyl-amino-5-methylisoxazole. MS (ES) m/z 279 (M⁺+1). The title compound was prepared in accordance with the general method of Example 6(b) using the intermediate 4-[N-(tetrahydro-2H-pyran-4-yl)]-N-trifluoroacetyl-amino-5-methylisoxazole (max 17.25 mmol), with the exception that the product was purified by flash chromatography (heptane/EtOAc 3:2), giving the title compound (3.03 g, 67%).

¹H NMR (CDCl₃, 300 MHz) δ 7.85 (s, 1H), 4.89-4.75 (m, 1H), 4.17-4.07 (m, 2H), 3.54-3.44 (m, 2H), 2.75-2.60 (m, 2H), 2.56 (s, 3H), 1.72-1.63 (m, 2H); MS (ES) m/z 263 (M+1).

### Example 9(b) (2E)-3-Dimethylamino-1-[1-(tetrahydro-2H-pyran-4-yl)-2-trifluoromethyl-1H-imidazol-5-yl]prop-2-en-1-one

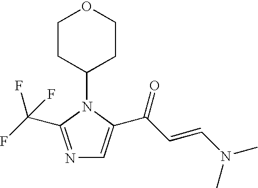

The title compound was prepared in accordance with the general method of Example 7(c) with the exception that the product was purified by flash chromatography (EtOAc). Using 5-acetyl-1-(tetrahydro-2H-pyran-4-yl)-2-trifluoromethyl-1H-imidazole (3.03 g, 11.55 mmol) the title compound was obtained (3.2 g, 87%).

¹H NMR (CDCl₃, 300 MHz) δ 7.72 (d, J=12.3 Hz, 1H), 7.49 (s, 1H), 5.50 (d, J=12.3 Hz, 1H), 4.89-4.75 (m, 1H), 4.14-4.05 (m, 2H), 3.54-3.44 (m, 2H), 3.16 (br. s, 3H), 2.93 (br. s, 3H), 2.86-2.72 (m, 2H), 1.80-1.72 (m, 2H); MS (ES) m/z 318 (M+1).

### Example 9(c) (2Z)-3-Dimethylamino-2-fluoro-1-[1-(tetrahydro-2H-pyran-4-yl)-2-trifluoromethyl-1H-imidazol-5-yl]prop-2-en-1-one

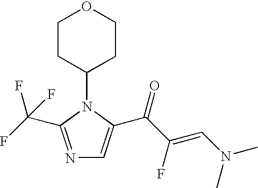

Selectfluor (0.370 g, 1.04 mmol) was added in portions to a stirred solution of (2E)-3-dimethylamino-1-[1-(tetrahydro-2H-pyran-4-yl)-2-trifluoromethyl-1H-imidazol-5-yl]prop-2-en-1-one (0.300 g, 0.946 mmol) in MeCN (20 mL) at 0° C. After stirring for 0.5 h at r.t. more Selectfluor (0.050 g, 0.14 mmol) was added, and the mixture was stirred for 0.5 h. The solvent was evaporated in vacuo, diluted with 3% aqueous NH₃ (20 mL) and extracted with CHCl₃ (3×20 mL). The organic extracts were dried (Na₂SO₄), evaporated in vacuo and the crude product was purified by flash chromatography (heptane/EtOAc 1:2), followed by neat EtOAc) to obtain the title compound (0.170 g, 53%).

¹H NMR (CDCl₃, 300 MHz) δ 7.34 (s, 1H), 6.85 (d, J=26.7 Hz, 1H), 4.67-4.54 (m, 1H), 4.11-4.03 (m, 2H), 3.50-3.38 (m, 2H), 3.14 (s, 6H), 2.72-2.56 (m, 2H), 1.83-1.74 (m, 2H); MS (ES) m/z 336 (M+1).

### Example 9(d) 5-Fluoro-4-[1-(tetrahydro-2H-pyran-4-yl)-2-(trifluoromethyl)-1H-imidazol-5-yl]pyrimidin-2-amine

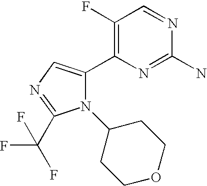

The title compound was prepared in accordance with the method in Example 7(e). Using (2Z)-3-dimethylamino-2-fluoro-1-[1-(tetrahydro-2H-pyran-4-yl)-2-trifluoromethyl-1H-imidazol-5-yl]prop-2-en-1-one (0.330 g, 1.0 mmol) and guanidine carbonate (0.45 g, 2.50 mmol). After purification by flash chromatography (heptane/EtOAc 1:2), the title compound was obtained (0.170 g, 51%).

¹H NMR (CDCl₃, 300 MHz) δ 8.29 (s, 1H), 7.63 (d, J=2.7 Hz, 1H), 5.10 (br.s., 2H), 4.88-4.76 (m, 1H), 4.16-4.07 (m, 2H), 3.53-3.42 (m, 2H), 2.80-2.65 (m, 2H), 1.89-1.81 (m, 2H); MS (ES) m/z 332 (M+1).

### Example 10

### 5-Fluoro-4-[2-methyl-1-(tetrahydro-2H-pyran-4-yl)-1H-imidazol-5-yl]-N-[6-(morpholin-4-ylmethyl)pyridin-3-yl]pyrimidin-2-amine

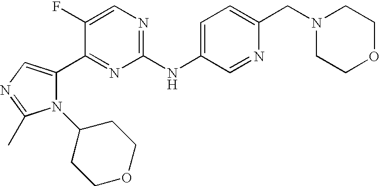

5-Fluoro-4-[2-methyl-1-(tetrahydro-2H-pyran-4-yl)-1H-imidazol-5-yl]pyrimidin-2-amine (obtained in Example 7) (50 mg, 180 μmol) and 4-[(5-bromopyridin-2-yl)methyl]morpholine (as described in WO 200190072) (46.4 mg, 180 μmol) in dry dioxane (2.3 mL) were purged with Ar (g) for 10 min. Pd₂(dba)₃ (8.3 mg, 9 μmol), X-Phos (8.6 mg, 18 μmol) and Cs₂CO₃ (102 mg, 289 μmol) were added and Ar (g) was bubbled through the mixture for 5 min prior to heating at 90° C. for 72 h. The mixture was allowed to cool, diluted with CH₂Cl₂ and filtered through diatomaceous earth. The organics were washed with water, dried (Na₂SO₄), filtered and concentrated. The crude was purified by flash silica gel chromatography EtOAc/MeOH 20:1-4:1, the residue was dissolved in CHCl₃ and filtered through tightly packed glass wool and concentrated to give 33 mg (40%) of the title compound.

¹H NMR (400 MHz, MeOD, 298 K) δ 8.75 (d, 1H), 8.45 (d, 1H), 8.11 (dd, 1H), 7.47-7.43 (m, 2H), 5.16 (m, 1H), 3.94 (m, 2H), 3.70 (t, 4H), 3.61 (s, 2H), 3.24 (m, 2H), 2.63 (s, 3H), 2.51 (t, 4H), 2.37 (m, 2H), 1.88 (m, 2H); MS (ES) m/z 454 (M+1).

### Example 11

### 5-({5-Fluoro-4-[2-methyl-1-(tetrahydro-2H-pyran-4-yl)-1H-imidazol-5-yl]pyrimidin-2-yl}amino)pyridine-2-carbaldehyde

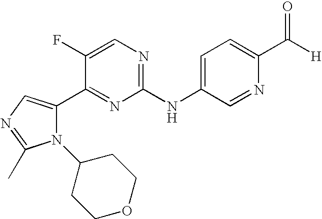

5-Fluoro-4-[2-methyl-1-(tetrahydro-2H-pyran-4-yl)-1H-imidazol-5-yl]pyrimidin-2-amine (obtained in Example 7) (1.3 g, 4.7 mmol) and 5-bromopyridine-2-carbaldehyde (872 mg, 4.69 mmol) in dry dioxane (60 mL) were purged with Ar (g) for 10 min. Pd₂(dba)₃ (258 mg, 281 μmol), X-Phos (268 mg, 562 μmol) and Cs₂CO₃ (2.9 g, 8.91 mmol) were added and Ar (g) was bubbled through the mixture for 5 min prior to heating at 90° C. for 68 h.

The mixture was allowed to cool, diluted with CH₂Cl₂ and filtered through diatomaceous earth and concentrated. The residue was re-dissolved in CH₂Cl₂ and the organics were washed with water, the aqueous phase was backwashed with CH₂Cl₂. The combined organics were dried (Na₂SO₄), filtered and concentrated. The crude was combined with that from another identical reaction, with the exception that different amounts of starting materials were used: 5-fluoro-4-[2-methyl-1-(tetrahydro-2H-pyran-4-yl)-1H-imidazol-5-yl]pyrimidin-2-amine (1.1 g, 3.97 mmol), 5-bromopyridine-2-carbaldehyde (738 mg, 3.97 mmol), dry dioxane (51 mL), Pd₂(dba)₃ (21 mg, 238 μmol), X-Phos (227 mg, 476 μmol) and Cs₂CO₃ (2.456 g, 7.54 mmol) and the reaction was heated at 90° C. for 45 h. The combined crudes were purified by flash silica gel chromatography EtOAc/MeOH 50:1-15:1, the residue was triturated with EtOAc/heptane to give 1.293 g (39%).

¹H NMR (400 MHz, DMSO-d₆, 298 K) δ 10.33 (s, 1H), 9.86 (m, 1H), 9.04 (d, 1H), 8.72 (d, 1H), 8.32 (dd, 1H), 7.91 (d, 1H), 7.39 (d, 1H), 5.01 (m, 1H), 3.83 (m, 2H), 3.15 (m, 2H), 2.56 (s, 3H), 2.20 (m, 2H), 1.84 (m, 2H); MS (ES) m/z 383 (M+1).

### Examples 12-29

The examples were prepared according to the following procedure: 5-({5-Fluoro-4-[2-methyl-1-(tetrahydro-2H-pyran-4-yl)-1H-imidazol-5-yl]pyrimidin-2-yl}amino)pyridine-2-carbaldehyde (obtained in Example 11) (50 mg, 130.8 μmol) was added to a deep well plate. Each amine (indicated for each example in turn) (1.5 equivalents) was added to it's corresponding well. Sodium triacetoxyborohydride (approx. 2-3 equivalents) followed by NMP (500 μL) was added to each well. The reactions were stirred at 21° C. for 70 h after which they were transferred to another deep well plate, diluted with NMP (300 μL) and purified by preparative chromatography.

Yields are approximate due to remaining salts and solvents after preparative chromatography; in particular a yield of 100% indicates the presence of salt or solvent in the sample in addition to the stated final compound.

Preparative chromatography was run on a Waters FractionLynx system with a Autosampler combined Automated Fraction Collector (Waters 2767), Gradient Pump (Waters 2525), Regeneration Pump (Waters 600), Make Up Pump (Waters 515), Waters Active Splitter, Column Switch (Waters CFO), PDA (Waters 2996) and Waters ZQ mass spectrometer. Column; XBridge™ Prep C8 5 μm OBD™ 19×100 mm, with guard column; XTerra® Prep MS C8 10 μm 19×10 mm Cartridge. A gradient from 100% A (95% 0.1M NH₄OAc in MilliQ water and 5% MeCN) to 100% B (100% MeCN) was applied for LC-separation at flow rate 25 ml/min. The PDA was scanned from 210-350 nm. The ZQ mass spectrometer was run with ESI in positive mode. The Capillary Voltage was 3 kV and the Cone Voltage was 30V. Mixed triggering, UV and MS signal, determined the fraction collection.

Purity analysis was run on a Water Acquity system with PDA (Waters 2996) and Waters ZQ mass spectrometer. Column; Acquity UPLC™ BEH C₈ 1.7 μm 2.1×50 mm. The column temperature was set to 65° C. A linear 2 min gradient from 100% A (A: 95% 0.01M NH₄OAc in MilliQ water and 5% MeCN) to 100% B (5% 0.01M NH₄OAc in MilliQ water and 95% MeCN) was applied for LC-separation at flow rate 1.2 ml/min. The PDA was scanned from 210-350 nm and 254 nm was extracted for purity determination. The ZQ mass spectrometer was run with ESI in pos/neg switching mode. The Capillary Voltage was 3 kV and the Cone Voltage was 30V.

### Example 12

### 5-Fluoro-4-[2-methyl-1-(tetrahydro-2H-pyran-4-yl)-1H-imidazol-5-yl]-N-[6-(piperidin-1-ylmethyl)pyridin-3-yl]pyrimidin-2-amine

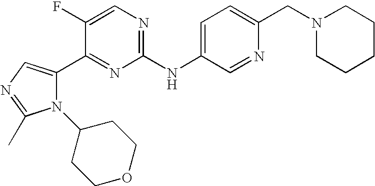

Amine: piperidine;

Yield (%): Ret. T (min): 0.66

¹H NMR (400 MHz, DMSO-d₆, 298 K) δ ppm 9.64 (s, 1H), 8.66 (d, 1H), 8.58 (d, 1H), 7.94 (dd, 1H), 7.35 (d, 1H), 7.32 (d, 1H), 5.00 (m, 1H), 3.79 (dd, 2H), 3.47 (s, 2H), 3.06 (m, 2H), 2.35 (m, 4H), 2.16 (m, 2H), 1.76 (m, 2H), 1.49 (m, 4H), 1.39 (m, 2H); m/z 452 (M+1).

### Example 13

### 5-Fluoro-N-{6-[(4-methyl-1,4-diazepan-1-yl)methyl]pyridin-3-yl}-4-[2-methyl-1-(tetrahydro-2H-pyran-4-yl)-1H-imidazol-5-yl]pyrimidin-2-amine

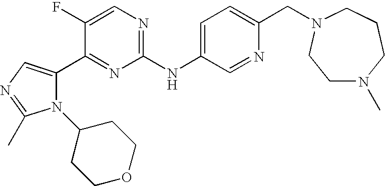

Amine: 1-methyl-1,4-diazepane

Yield (%): 37; Ret. T (min): 0.62

¹H NMR (400 MHz, DMSO-d₆, 298 K) δ ppm 9.63 (s, 1H), 8.66 (d, 1H), 8.58 (d, 1H), 7.95 (dd, 1H), 7.35 (m, 2H), 5.00 (m, 1H), 3.80 (dd, 2H), 3.65 (s, 2H), 3.08 (m, 2H), 2.66 (m, 4H), 2.56 (m, 2H, partially obscured by DMSO), 2.24 (s, 3H), 2.16 (m, 2H), 1.67-1.80 (m, 4H); m/z 481 (M+1).

### Example 14

### 5-Fluoro-4-[2-methyl-1-(tetrahydro-2H-pyran-4-yl)-1H-imidazol-5-yl]-N-{6-[(4 pyrimidin-2-ylpiperazin-1-yl)methyl]pyridin-3-yl}pyrimidin-2-amine

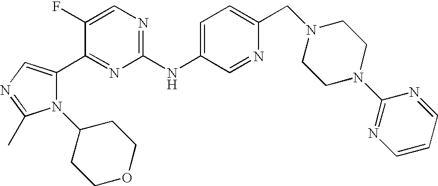

Amine: 2-piperazin-1-ylpyrimidine

Yield (%): 60; Ret. T (min): 0.82

¹H NMR (400 MHz, DMSO-d₆, 298 K) δ ppm 9.67 (s, 1H), 8.70 (d, 1H), 8.60 (d, 1H), 8.35 (d, 2H), 7.98 (dd, 1H), 7.37 (m, 2H), 6.61 (t, 1H), 5.01 (m, 1H), 3.81 (dd, 2H), 3.73 (m, 4H), 3.58 (s, 2H), 3.07 (m, 2H), 2.47 (m, 4H), 2.16 (m, 2H), 1.78 (m, 2H) m/z 531 (M+1).

### Example 15

### 5-Fluoro-N-(6-{[(2S)-2-(methoxymethyl)pyrrolidin-1-yl]methyl}pyridin-3-yl)-4-[2-methyl-1-(tetrahydro-2H-pyran-4-yl)-1H-imidazol-5-yl]pyrimidin-2-amine

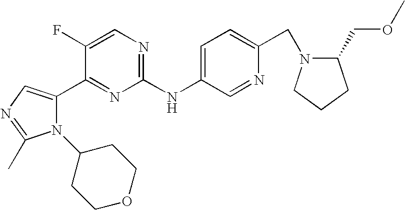

Amine: (2S)-2-(methoxymethyl)pyrrolidine

Yield (%): 100; Ret. T (min): 0.70

¹H NMR (400 MHz, DMSO-d₆, 298 K) δ ppm 9.62 (s, 1H), 8.66 (d, 1H), 8.58 (d, 1H), 7.93 (dd, 1H), 7.35 (d, 1H), 7.31 (d, 1H), 5.00 (m, 1H), 4.06 (d, 1H), 3.79 (dd, 2H), 3.47 (d, 1H), 3.24 (s, 3H), 3.06 (m, 2H), 2.84 (m, 1H), 2.75 (m, 1H), 2.14-2.26 (m, 3H), 1.85 (m, 2H), 1.76 (m, 2H), 1.63 (m, 2H), 1.50 (m, 1H); m/z 482 (M+1).

### Example 16

### N-{6-[(4-Acetyl-1,4-diazepan-1-yl)methyl]pyridin-3-yl}-5-fluoro-4-[2-methyl-1-(tetrahydro-2H-pyran-4-yl)-1H-imidazol-5-yl]pyrimidin-2-amine

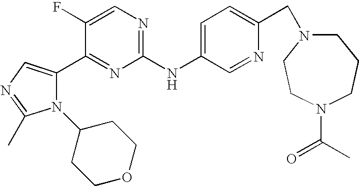

Amine: 1-(1,4-diazepan-1-yl)ethanone:

Yield (%): 61; Ret. T (min): 0.68

m/z 509 (M+1).

### Example 17

### N-{6-[(2,6-Dimethylmorpholin-4-yl)methyl]pyridin-3-yl}-5-fluoro-4-[2-methyl-1-(tetrahydro-2H-pyran-4-yl)-1H-imidazol-5-yl]pyrimidin-2-amine

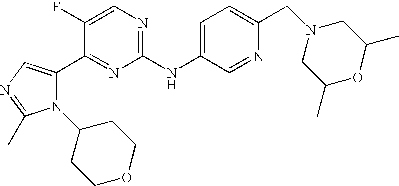

Amine: 2,6-dimethylmorpholine

Yield (%): 100; Ret. T (min): 0.81 m/z 482 (M+1).

### Example 18

### N-{6-[(4,4-Difluoropiperidin-1-yl)methyl]pyridin-3-yl}-5-fluoro-4-[2-methyl-1-(tetrahydro-2H-pyran-4-yl)-1H-imidazol-5-yl]pyrimidin-2-amine

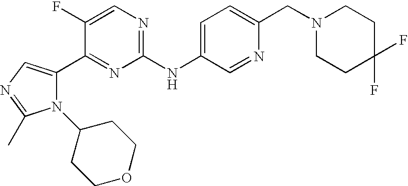

Amine: 4,4-difluoropiperidine

Yield (%): 77; Ret. T (min): 0.90

¹H NMR (400 MHz, DMSO-d₆, 298 K) δ ppm 9.67 (s, 1H), 8.69 (d, 1H), 8.59 (d, 1H), 7.97 (dd, 1H), 7.34-7.35 (m, 2H), 5.00 (m, 1H), 3.80 (dd, 2H), 3.60 (s, 2H), 3.07 (m, 2H), 2.16 (m, 2H), 1.91-2.00 (m, 4H), 1.76 (m, 2H); m/z 488 (M+1).

### Example 19

### 5-Fluoro-4-[2-methyl-1-(tetrahydro-2H-pyran-4-yl)-1H-imidazol-5-yl]-N-[6-(pyrrolidin-1-ylmethyl)pyridin-3-yl]pyrimidin-2-amine

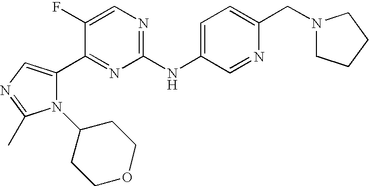

Amine: pyrrolidine

Yield (%): 55; Ret. T (min): 0.61

¹H NMR (400 MHz, DMSO-d₆, 298 K) δ ppm 9.63 (s, 1H), 8.66 (d, 1H), 8.58 (d, 1H), 7.93 (dd, 1H), 7.35 (d, 1H), 7.32 (d, 1H), 5.01 (m, 1H), 3.79 (dd, 2H), 3.63 (s, 2H), 3.05 (m, 2H), 2.46 (m, 4H), 2.16 (m, 2H), 1.76 (m, 2H), 1.69 (m, 4H); m/z 438 (M+1).

### Example 20

### N-[6-({[(6-Chloropyridin-3-yl)methyl]amino}methyl)pyridin-3-yl]-5-fluoro-4-[2-methyl-1-(tetrahydro-2H-pyran-4-yl)-1H-imidazol-5-yl]pyrimidin-2-amine

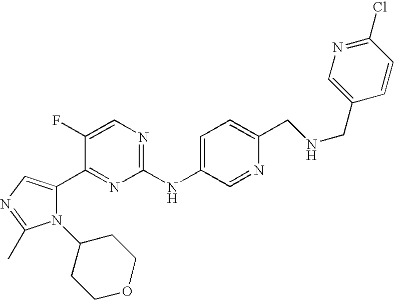

Amine: (6-chloropyridin-3-yl)methanamine

Yield (%) 50; Ret T (min): 0.79

¹H NMR (400 MHz, DMSO-d₆, 298 K) δ ppm 9.63 (s, 1H), 8.68 (d, 1H), 8.58 (d, 1H), 8.36 (d, 1H), 7.95 (dd, 1H), 7.84 (dd, 1H), 7.46 (d, 1H), 7.34-7.37 (m, 2H), 5.00 (m, 1H), 3.80 (dd, 2H), 3.72 (m 4H), 3.06 (m, 2H), 2.15 (m, 2H), 1.76 (m, 2H); m/z 509 (M+1).

### Example 21

### 5-Fluoro-4-[2-methyl-1-(tetrahydro-2H-pyran-4-yl)-1H-imidazol-5-yl]-N-[6-(1,4 oxazepan-4-ylmethyl)pyridin-3-yl]pyrimidin-2-amine

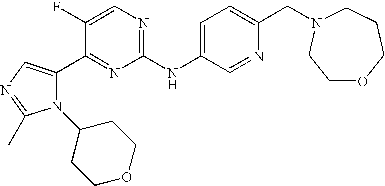

Amine: 1,4-oxazepane

Yield (%): 39; Ret T (min): 0.68

¹H NMR (400 MHz, DMSO-d₆, 298 K) δ ppm 9.64 (s, 1H), 8.67 (d, 1H), 8.58 (d, 1H), 7.96 (dd, 1H), 7.34-7.38 (m, 2H), 5.00 (m, 1H), 3.80 (dd, 2H), 3.68-3.71 (m, 4H), 3.60 (m, 2H), 3.08 (m, 2H), 2.65 (m, 4H), 2.16 (m, 2H), 1.74-1.83 (m, 4H); m/z 468 (M+1).

### Example 22

### 5-Fluoro-N-{6-[(4-methoxypiperidin-1-yl)methyl]pyridin-3-yl}-4-[2-methyl-1-(tetrahydro-2H-pyran-4-yl)-1H-imidazol-5-yl]pyrimidin-2-amine

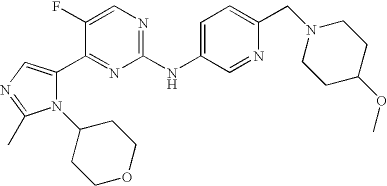

Amine: 4-methoxypiperidine

Yield (%): 76; Ret. T (min): 0.68

¹H NMR (400 MHz, DMSO-d₆, 298 K) δ ppm 9.64 (s, 1H), 8.66 (d, 1H), 8.58 (d, 1H), 7.95 (dd, 1H), 7.30-7.35 (m, 2H), 5.00 (m, 1H), 3.79 (dd, 2H), 3.49 (s, 2H), 3.21 (s, 3H), 3.16 (m, 1H), 3.05 (m, 2H), 2.66 (m, 2H), 2.09-2.21 (m, 4H), 1.82 (m, 2H), 1.76 (m, 2H), 1.41 (m, 2H); m/z 482 (M+1).

### Example 23

### (1-{[5-({5-Fluoro-4-[2-methyl-1-(tetrahydro-2H-pyran-4-yl)-1H-imidazol-5-yl]pyrimidin-2-yl}amino)pyridin-2-yl]methyl}piperidin-3-yl)methanol

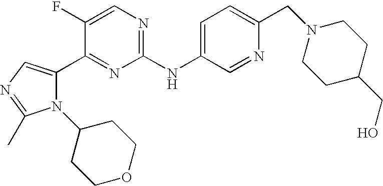

Amine: 3-piperidylmethanol

Yield (%): 60; Ret. T (min): 0.62

¹H NMR (400 MHz, DMSO-d₆, 298 K) δ ppm 9.63 (s, 1H), 8.66 (d, 1H), 8.58 (d, 1H), 7.94 (dd, 1H), 7.35 (d, 1H), 7.33 (d, 1H), 4.99 (m, 1H), 4.36 (t, 1H), 3.80 (dd, 2H), 3.49 (dd, 2H), 3.15-3.27 (m, 2H), 3.07 (m, 2H), 2.84 (m, 1H), 2.70-2.72 (m, 1H), 2.53 (s, 3H), 2.16 (m, 2H), 1.95 (m, 1H), 1.76 (m, 2H), 1.57-1.70 (m, 4H), 1.45 (m, 1H), 0.88 (m, 1H); m/z 482 (M+1).

### Example 24

### 1-[3-({[5-({5-Fluoro-4-[2-methyl-1-(tetrahydro-2H-pyran-4-yl)-1H-imidazol-5-yl]pyrimidin-2-yl}amino)pyridin-2-yl]methyl}amino)propyl]pyrrolidin-2-one

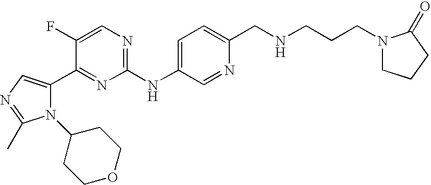

Amine: 1-(3-aminopropyl)pyrrolidin-2-one

Yield (%): 72; Ret. T (min): 0.61 m/z 509 (M+1).

### Example 25

### 5-Fluoro-4-[2-methyl-1-(tetrahydro-2H-pyran-4-yl)-1H-imidazol-5-yl]-N-{6-[(4 pyrrolidin-1-ylpiperidin-1-yl)methyl]pyridin-3-yl}pyrimidin-2-amine

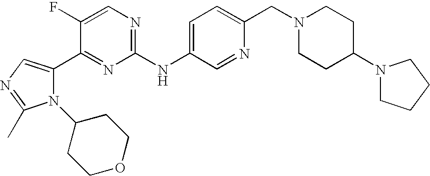

Amine: 4-pyrrolidin-1-ylpiperidine

Yield (%): 53; Ret. T (min): 0.64

¹H NMR (400 MHz, DMSO-d₆, 298 K) δ ppm 9.64 (s, 1H), 8.66 (d, 1H), 8.58 (d, 1H), 7.95 (dd, 1H), 7.35 (d, 1H), 7.32 (d, 1H), 5.00 (m, 1H), 3.79 (m, 2H), 3.48 (s, 2H), 3.05 (m, 2H), 2.78 (m, 2H), 2.45 (br. s., 4H), 2.16 (m, 2H), 1.92-2.03 (m, 3H), 1.77 (m, 4H), 1.65 (br. s., 4H), 1.39 (m, 2H); m/z 521 (M+1).

### Example 26

### 3-[{[5-({5-Fluoro-4-[2-methyl-1-(tetrahydro-2H-pyran-4-yl)-1H-imidazol-5-yl]pyrimidin-2-yl}amino)pyridin-2-yl]methyl}(tetrahydrofuran-2-ylmethyl)amino]propanenitrile

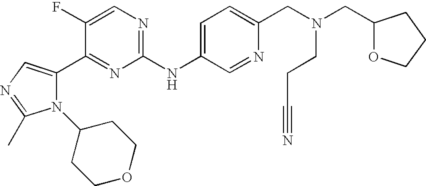

Amine: 3-(oxolan-2-ylmethylamino)propanenitrile

Yield (%): 64; Ret. T (min): 0.86

¹H NMR (400 MHz, DMSO-d₆, 298 K) δ ppm 9.65 (s, 1H), 8.69 (d, 1H), 8.58 (d, 1H), 7.97 (dd, 1H), 7.41 (d, 1H), 7.34 (d, 1H), 5.00 (m, 1H), 3.92 (m, 1H), 3.78-3.84 (m, 3H), 3.68-3.75 (m, 2H), 3.60 (m, 1H), 3.11 (m, 2H), 2.76-2.88 (m, 2H), 2.64-2.68 (m, 2H), 2.56-2.58 (m, 2H), 2.17 (m, 2H), 1.84-1.92 (m, 1H), 1.73-1.78 (m, 4H), 1.42-1.51 (m, 1H); m/z 521 (M+1).

### Example 27

### N-[6-(Azetidin-1-ylmethyl)pyridin-3-yl]-5-fluoro-4-[2-methyl-1-(tetrahydro-2H-pyran-4-yl)-1H-imidazol-5-yl]pyrimidin-2-amine

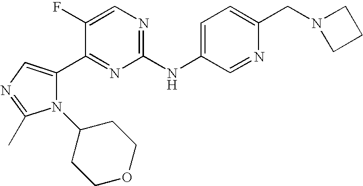

Amine: azetidine

Yield (%): 44; Ret. T (min): 0.59 m/z 424 (M+1).

### Example 28

### N-(6-{[Ethyl(2-methoxyethyl)amino]methyl}pyridin-3-yl)-5-fluoro-4-[2-methyl-1-(tetrahydro-2H-pyran-4-yl)-1H-imidazol-5-yl]pyrimidin-2-amine

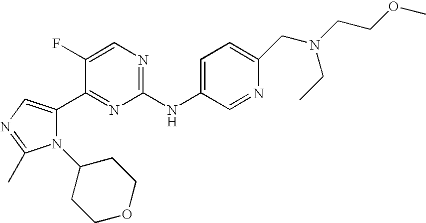

Amine: N-(2-methoxyethyl)ethanamine

Yield (%): 46; Ret. T (min): 0.71

¹H NMR (400 MHz, DMSO-d₆, 298 K) δ ppm 9.62 (s, 1H), 8.66 (d, 1H), 8.58 (d, 1H), 7.94 (dd, 1H), 7.34 (s, 1H), 7.36 (d, 1H), 5.00 (m, 1H), 3.80 (dd, 2H), 3.64 (s, 2H), 3.41 (t, 2H), 3.21 (s, 3H), 3.08 (m, 2H), 2.62 (t, 2H), 2.16 (m, 2H), 1.76 (m, 2H), 0.98 (t, 3H); m/z 470 (M+1).

### Example 29

### ({[5-({5-Fluoro-4-[2-methyl-1-(tetrahydro-2H-pyran-4-yl)-1H-imidazol-5-yl]pyrimidin-2-yl}amino)pyridin-2-yl]methyl}amino)acetonitrile

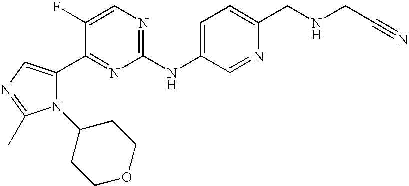

Amine: 2-aminoacetonitrile

Yield (%): 42; Ret. T (min): 0.67

¹H NMR (400 MHz, DMSO-d₆, 298 K) δ ppm 9.66 (s, 1H), 8.70 (d, 1H), 8.59 (d, 1H), 7.96 (dd, 1H), 7.31-7.35 (m, 2H), 5.00 (m, 1H), 3.79-3.83 (m, 4H), 3.65 (m, 2H), 3.06 (m, 2H), 2.16 (m, 2H), 1.76 (m, 2H); m/z 423 (M+1).

### Example 30

### {5-Fluoro-4-[2-methyl-3-(tetrahydro-pyran-4-yl)-3H-imidazol-4-yl]-pyrimidin-2-yl}-isoquinolin-4-yl-amine

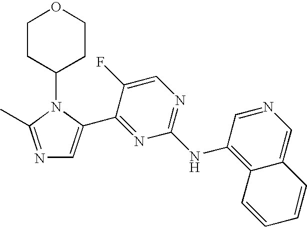

The title compound was prepared in accordance with the general method A using 5-fluoro-4-[2-methyl-1-(tetrahydro-2H-pyran-4-yl)-1H-imidazol-5-yl]pyrimidin-2-amine (obtained in Example 7) (50 mg, 0.18 mmol) and 4-bromo-isoquinoline (37 mg, 0.18 mmol) to give the title compound (11 mg, 15%).

¹H NMR (CDCl₃) δ ppm 9.08 (s, 1H) 8.65 (s, 1H) 8.41 (d, J=3.03 Hz, 1H) 8.14 (d, J=7.83 Hz, 1H) 8.06 (d, J=8.34 Hz, 1H) 7.77 (m, 1H) 7.70 (m, 1H) 7.43 (m, 1H) 4.96 (m, 1H) 3.63 (m, 2H) 2.64 (m, 2H) 2.49 (s, 3H) 2.14-2.04 (m, 2H), 1.39 (m, 2H); MS (ES) m/z 405 (M+1).

### Example 31

### {5-Fluoro-4-[2-methyl-3-(tetrahydro-pyran-4-yl)-3H-imidazol-4-yl]-pyrimidin-2-yl}-pyridin-4-yl-amine

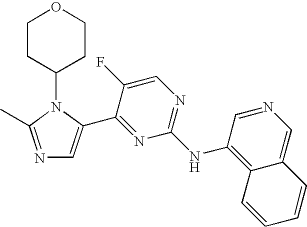

The title compound was prepared in accordance with the general method A using 5-fluoro-4-[2-methyl-1-(tetrahydro-2H-pyran-4-yl)-1H-imidazol-5-yl]pyrimidin-2-amine (obtained in Example 7) (50 mg, 0.18 mmol) and 4-bromopyridine (35 mg, 0.18 mmol) to give the title compound (29 mg, 45%) which was later transformed to the hydrochloride salt as described in general method A.

¹H NMR (CDCl₃) δ ppm 8.44 (m, 2H) 8.38 (d, J=2.78 Hz, 1H) 7.91 (br s, 1H) 7.66 (d, J=4.04 Hz, 1H) 7.54 (m, 2H) 5.10 (m, 1H) 4.07 (m, 2H) 3.33 (m, 2H) 2.65 (s, 3H) 2.50 (m, 2H) 1.88 (m, 2H); MS (ES) m/z 355 (M+1).

### Example 32

### 2-Bromo-5-fluoro-4-[2-methyl-1-(tetrahydro-2H-pyran-4-yl)-1H-imidazol-5-yl]pyrimidine

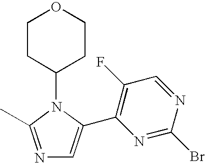

Trimethylsilyl bromide (6.4 mL, 49 mmol) was added dropwise to 5-fluoro-4-[2-methyl-1-(tetrahydro-2H-pyran-4-yl)-1H-imidazol-5-yl]pyrimidin-2-amine (1.5 g, 5.4 mmol) in CH₂Br₂ (60 mL) under an argon atmosphere followed by addition of t-Butylnitrite (12 mL, 100 mmol). The reaction was stirred at r.t. for 5 hours before sat NaHCO₃ (aq): H₂O (1:1, 100 mL) and CH₂Cl₂ (50 mL) was added. The mixture was extracted and the aqueous phase was washed with CH₂Cl₂ (2×50 mL). The organic phases were combined, dried and the solvent was evaporated in vacuo to give the title product (1.48 g, 80%).

¹H NMR (400 MHz, CDCl₃) δ ppm 8.44 (d, 1H) 7.83 (d, 1H) 5.42-5.52 (m, 1H) 4.18 (dd, 2H) 3.52-3.63 (m, 2H) 2.75 (s, 3H) 2.35-2.49 (m, 2H) 1.96-2.05 (m, 2H); MS (ESI) m/z 341/343 (M+1).

### Example 33

### tert-Butyl 4-({5-fluoro-4-[2-methyl-1-(tetrahydro-2H-pyran-4-yl)-1H-imidazol-5-yl]pyrimidin-2-yl}amino)piperidine-1-carboxylate

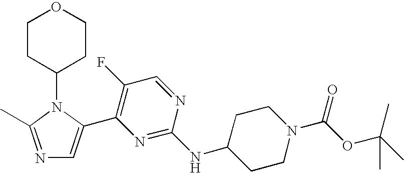

The title compound was prepared in accordance with the general method B using 2-bromo-5-fluoro-4-[2-methyl-1-(tetrahydro-2H-pyran-4-yl)-1H-imidazol-5-yl]pyrimidine (obtained in Example 32) (900 mg, 2.64 mmol) and tert-butyl 4-aminopiperidine-1-carboxylate (1.1 g, 5.7 mmol) to give the title compound (560 mg, 46%).

¹H NMR (400 MHz, CDCl₃) δ ppm 8.17 (d, 1H) 7.51 (d, 1H) 5.07-5.20 (m, 1H) 4.98 (d, 1H) 4.13 (dd, 2H) 3.85-4.10 (m, 3H) 3.41-3.50 (m, 2H) 2.89 (t, 2H) 2.63 (s, 3H) 2.40-2.55 (m, 2H) 1.99-2.08 (m, 2H) 1.85-1.92 (m, 7H) 1.46 (s, 9H) 1.38-1.44 (m, 1H); MS (ESI) m/z 461 (M+1).

### Example 34

### 5-Fluoro-4-[2-methyl-1-(tetrahydro-2H-pyran-4-yl)-1H-imidazol-5-yl]-N-(tetrahydro-2H-pyran-4-yl)pyrimidin-2-amine

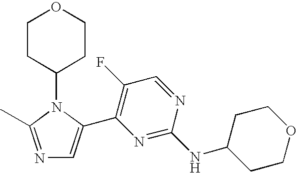

The title compound was prepared in accordance with the general method B using 2-bromo-5-fluoro-4-[2-methyl-1-(tetrahydro-2H-pyran-4-yl)-1H-imidazol-5-yl]pyrimidine (obtained in Example 32) (40 mg, 0.117 mmol) and tetrahydro-2H-pyran-4-amine hydrochloride (32 mg, 0.234 mmol) to give the title compound (25 mg, 59%).

¹H NMR (400 MHz, CDCl₃) δ ppm 8.18 (d, 1H) 7.54 (br. s., 1H) 5.04 (d, 1H) 4.15 (dd, 2H) 3.97-4.04 (m, 2H) 3.43-3.53 (m, 4H) 2.61-2.67 (m, 3H) 2.41-2.56 (m, 2H) 2.01 (s, 3H) 1.90 (dd, 2H) 1.49-1.65 (m, 2H); MS (ESI) m/z 362 (M+1).

### Example 35

### N-(1-Acetylpiperidin-4-yl)-5-fluoro-4-[2-methyl-1-(tetrahydro-2H-pyran-4-yl)-1H-imidazol-5-yl]pyrimidin-2-amine

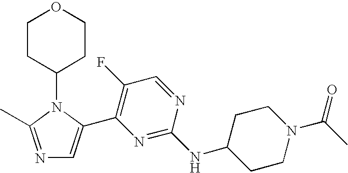

The title compound was prepared in accordance with the general method C using tert-butyl 4-({5-fluoro-4-[2-methyl-1-(tetrahydro-2H-pyran-4-yl)-1H-imidazol-5-yl]pyrimidin-2-yl}amino)piperidine-1-carboxylate (obtained in Example 33) (54 mg, 0.117 mmol) and acetyl chloride (8.5 μL, 0.117 mmol) to give the title compound (38 mg, 81%).

¹H NMR (400 MHz, CDCl₃) δ ppm 8.17 (d, 1H) 4.99-5.15 (m, 2H) 4.48 (d, 1H) 4.12 (dd, 2H) 3.93-4.05 (m, 1H) 3.76-3.85 (m, 1H) 3.45 (t, 2H) 3.12-3.23 (m, 1H) 2.77-2.87 (m, 1H) 2.62 (s, 3H) 2.42-2.55 (m, 2H) 2.10 (s, 3H) 2.03-2.16 (m, 2H) 1.84-1.91 (m, 2H) 1.35-1.51 (m, 2H); MS (ESI) m/z 403 (M+1).

### Example 36

### N-Cyclohexyl-5-fluoro-4-[2-methyl-1-(tetrahydro-2H-pyran-4-yl)-1H-imidazol-5-yl]pyrimidin-2-amine

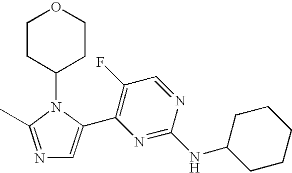

The title compound was prepared in accordance with the general method B using 2-bromo-5-fluoro-4-[2-methyl-1-(tetrahydro-2H-pyran-4-yl)-1H-imidazol-5-yl]pyrimidine (obtained in Example 32) (40 mg, 0.117 mmol) and cyclohexylamine (27 μL, 0.24 mmol) to give the title compound (28 mg, 67%).

¹H NMR (400 MHz, CDCl₃) δ ppm 8.15 (d, 1H) 7.51 (d, 1H) 5.20-5.33 (m, 1H) 5.03-5.12 (m, 1H) 4.14 (dd, 2H) 3.43-3.54 (m, 2H) 2.64 (s, 3H) 2.37-2.51 (m, 2H) 1.99-2.07 (m, 2H) 1.88-1.96 (m, 2H) 1.72-1.81 (m, 2H) 1.58-1.68 (m, 1H) 1.15-1.40 (m, 5H); MS (ESI) m/z 360 (M+1).

### Example 37

### N-(1-Benzylpiperidin-4-yl)-5-fluoro-4-[2-methyl-1-(tetrahydro-2H-pyran-4-yl)-1H-imidazol-5-yl]pyrimidin-2-amine

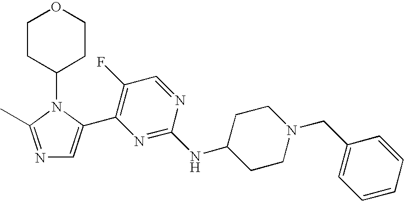

The title compound was prepared in accordance with the general method B using 2-bromo-5-fluoro-4-[2-methyl-1-(tetrahydro-2H-pyran-4-yl)-1H-imidazol-5-yl]pyrimidine (obtained in Example 32) (40 mg, 0.117 mmol) and 1-benzylpiperidin-4-amine (50 μL, 0.25 mmol) to give the title compound (30 mg, 57%).

¹H NMR (400 MHz, CDCl₃) δ ppm 8.16 (d, 1H) 7.52 (d, 1H) 7.25-7.37 (m, 5H) 5.16-5.28 (m, 1H) 5.01 (d, 1H) 4.11 (dd, 2H) 3.70-3.86 (m, 1H) 3.56 (s, 2H) 3.40-3.52 (m, 2H) 2.87 (d, 2H) 2.64 (s, 3H) 2.37-2.53 (m, 2H) 2.16 (br. s., 2H) 2.00-2.08 (m, 2H) 1.85-1.93 (m, 2H) 1.53-1.68 (m, 2H); MS (ESI) m/z 451 (M+1).

### Example 38

### N-(1-Benzoylpiperidin-4-yl)-5-fluoro-4-[2-methyl-1-(tetrahydro-2H-pyran-4-yl)-1H-imidazol-5-yl]pyrimidin-2-amine

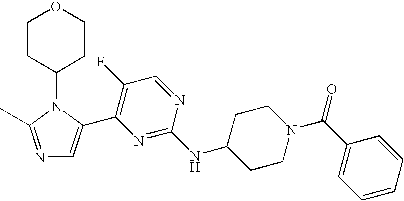

The title compound was prepared in accordance with the general method C using tert-butyl 4-({5-fluoro-4-[2-methyl-1-(tetrahydro-2H-pyran-4-yl)-1H-imidazol-5-yl]pyrimidin-2-yl}amino)piperidine-1-carboxylate (obtained in Example 33) (45 mg, 0.1 mmol) and benzoyl chloride (11.5 μL, 0.1 mmol) to give the title compound (26 mg, 57%).

¹H NMR (400 MHz, CDCl₃) δ ppm 8.18 (d, 1H) 7.53 (br. s., 1H) 7.36-7.45 (m, 5H) 5.04-5.17 (m, 2H) 4.61 (br. s., 1H) 4.14 (dd, 2H) 3.98-4.10 (m, 1H) 3.78 (br. s., 1H) 3.40-3.53 (m, 2H) 3.09 (br. s., 2H) 2.63 (s, 3H) 2.42-2.56 (m, 2H) 2.17 (br. s., 2H) 1.88 (dd, 2H) 1.37-1.62 (m, 2H); MS (ESI) m/z 465 (M+1).

### Example 39

### 5-Fluoro-4-[2-methyl-1-(tetrahydro-2H-pyran-4-yl)-1H-imidazol-5-yl]-N-[1-(phenylacetyl)piperidin-4-yl]pyrimidin-2-amine

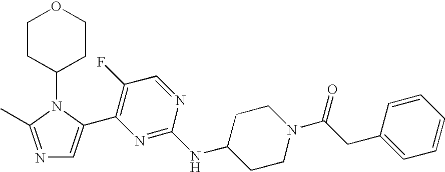

The title compound was prepared in accordance with the general method C using tert-butyl 4-({5-fluoro-4-[2-methyl-1-(tetrahydro-2H-pyran-4-yl)-1H-imidazol-5-yl]pyrimidin-2-yl}amino)piperidine-1-carboxylate (obtained in Example 33) (45 mg, 0.1 mmol) and phenylacetyl chloride (13 μL, 0.1 mmol) to give the title compound (27 mg, 58%).

¹H NMR (400 MHz, CDCl₃) δ ppm 8.16 (d, 1H) 7.52 (d, 1H) 7.19-7.39 (m, 5H) 4.93-5.03 (m, 1H) 4.51 (d, 1H) 4.12 (dd, 2H) 3.80-4.01 (m, 2H) 3.76 (s, 2H) 3.44 (t, 2H) 3.03-3.18 (m, 1H) 2.80-2.92 (m, 1H) 2.64 (s, 3H) 2.39-2.55 (m, 2H) 1.83-2.12 (m, 4H) 1.33-1.49 (m, 1H) 1.07-1.21 (m, 1H); MS (ESI) m/z 479 (M+1).

### Example 40

### Benzyl 4-({5-fluoro-4-[2-methyl-1-(tetrahydro-2H-pyran-4-yl)-1H-imidazol-5-yl]pyrimidin-2-yl}amino)piperidine-1-carboxylate

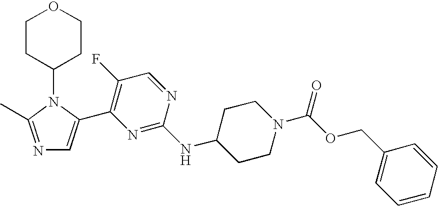

The title compound was prepared in accordance with the general method C using tert-butyl 4-({5-fluoro-4-[2-methyl-1-(tetrahydro-2H-pyran-4-yl)-1H-imidazol-5-yl]pyrimidin-2-yl}amino)piperidine-1-carboxylate (obtained in Example 33) (45 mg, 0.1 mmol) and benzyl chloroformate (14 μL, 0.1 mmol) to give the title compound (21 mg, 43%).

¹H NMR (400 MHz, CDCl₃) δ ppm 8.18 (d, 1H) 7.53 (br. s., 1H) 7.29-7.42 (m, 5H) 4.98-5.20 (m, 4H) 4.14 (dd, 4H) 3.86-4.02 (m, 1H) 3.38-3.54 (m, 2H) 2.90-3.12 (m, 2H) 2.64 (s, 3H) 2.42-2.55 (m, 2H) 2.00-2.11 (m, 2H) 1.85-1.94 (m, 2H) 1.45 (d, 2H); MS (ESI) m/z 495 (M+1).

### Example 41

### 5-Fluoro-N-[1-(methylsulfonyl)piperidin-4-yl]-4-[2-methyl-1-(tetrahydro-2H-pyran-4-yl)-1H-imidazol-5-yl]pyrimidin-2-amine

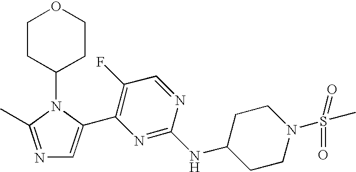

The title compound was prepared in accordance with the general method D using tert-butyl 4-({5-fluoro-4-[2-methyl-1-(tetrahydro-2H-pyran-4-yl)-1H-imidazol-5-yl]pyrimidin-2-yl}amino)piperidine-1-carboxylate (obtained in Example 33) (45 mg, 0.1 mmol) and methanesulfonyl chloride (8 μL, 0.1 mmol) to give the title compound (20 mg, 47%).

¹H NMR (400 MHz, CDCl₃) δ ppm 8.18 (d, 1H) 7.53 (d, 1H) 5.02-5.13 (m, 2H) 4.13 (dd, 2H) 3.87-3.98 (m, 1H) 3.70-3.79 (m, 2H) 3.41-3.51 (m, 2H) 2.86-2.96 (m, 2H) 2.81 (s, 3H) 2.63 (s, 3H) 2.44-2.57 (m, 2H) 2.12-2.22 (m, 2H) 1.88 (dd, 2H) 1.60-1.73 (m, 2H); MS (ESI) m/z 439 (M+1).

### Example 42

### 5-Fluoro-4-[2-methyl-1-(tetrahydro-2H-pyran-4-yl)-1H-imidazol-5-yl]-N-[1-(trifluoroacetyl)piperidin-4-yl]pyrimidin-2-amine

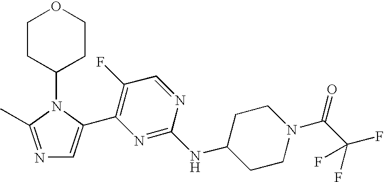

The title compound was isolated as a side product from Example 41 (10 mg, 22%).

¹H NMR (400 MHz, CDCl₃) δ ppm 8.20 (d, 1H) 7.56 (d, 1H) 4.98-5.13 (m, 2H) 4.45 (d, 1H) 4.15 (dd, 2H) 3.97-4.12 (m, 2H) 3.47 (t, 2H) 3.24-3.35 (m, 1H) 2.99-3.10 (m, 1H) 2.65 (s, 3H) 2.46-2.59 (m, 2H) 2.15-2.25 (m, 2H) 1.85-1.93 (m, 2H) 1.48-1.62 (m, 2H); MS (ESI) m/z 457 (M+1).

### Example 43

### 5-Fluoro-4-[2-methyl-1-(tetrahydro-2H-pyran-4-yl)-1H-imidazol-5-yl]-N-[1-(phenylsulfonyl)piperidin-4-yl]pyrimidin-2-amine

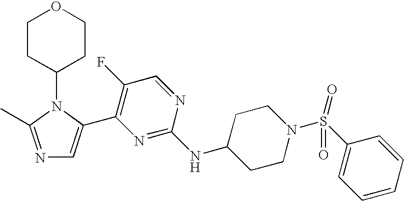

The title compound was prepared in accordance with the general method D using tert-butyl 4-({5-fluoro-4-[2-methyl-1-(tetrahydro-2H-pyran-4-yl)-1H-imidazol-5-yl]pyrimidin-2-yl}amino)piperidine-1-carboxylate (obtained in Example 33) (45 mg, 0.1 mmol) and benzenesulfonyl chloride (12.5 μL, 0.1 mmol) to give the title compound (35 mg, 71%).

¹H NMR (400 MHz, CDCl₃) δ ppm 8.12 (d, 1H) 7.72-7.81 (m, 2H) 7.60-7.68 (m, 1H) 7.56 (t, 2H) 7.50 (d, 2H) 4.98-5.09 (m, 2H) 3.94 (d, 2H) 3.63-3.77 (m, 3H) 3.34 (t, 2H) 2.61 (s, 3H) 2.45-2.55 (m, 2H) 2.33-2.44 (m, 2H) 2.06-2.15 (m, 2H) 1.76-1.85 (m, 2H) 1.58-1.72 (m, 2H); MS (ESI) m/z 501 (M+1).

### Example 44

### N-[1-(Benzylsulfonyl)piperidin-4-yl]-5-fluoro-4-[2-methyl-1-(tetrahydro-2H-pyran-4-yl)-1H-imidazol-5-yl]pyrimidin-2-amine

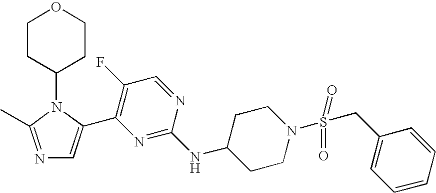

The title compound was prepared in accordance with the general method D using tert-butyl 4-({5-fluoro-4-[2-methyl-1-(tetrahydro-2H-pyran-4-yl)-1H-imidazol-5-yl]pyrimidin-2-yl}amino)piperidine-1-carboxylate (obtained in Example 33) (45 mg, 0.1 mmol) and phenylmethanesulfonyl chloride (19 mg, 0.1 mmol) to give the title compound (28 mg, 56%).

¹H NMR (400 MHz, CDCl₃) δ ppm 8.16 (d, 1H) 7.51 (d, 1H) 7.35-7.44 (m, 5H) 5.00-5.13 (m, 1H) 4.93 (d, 1H) 4.24 (s, 2H) 4.11 (dd, 2H) 3.73-3.87 (m, 1H) 3.61 (d, 2H) 3.37-3.49 (m, 2H) 2.68-2.77 (m, 2H) 2.63 (s, 3H) 2.40-2.54 (m, 2H) 1.95-2.05 (m, 2H) 1.82-1.90 (m, 2H) 1.39-1.52 (m, 2H); MS (ESI) m/z 515 (M+1).

### Pharmaceutical Formulations

According to one aspect of the present invention there is provided a pharmaceutical formulation comprising the compound of formula (I) as a free base or a pharmaceutically acceptable salt thereof, in an essentially pure and isolated form, for use in the prevention and/or treatment of conditions associated with glycogen synthase kinase-3.

The formulation used in accordance with the present invention may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream, for rectal administration as a suppository and for local administration in a body cavity or in a bone cavity.

The formulation may be in a form suitable for oral administration, for example as a tablet, for parenteral injection as a sterile solution or suspension. In general the above formulation may be prepared in a conventional manner using pharmaceutically carriers or diluents.

Suitable daily doses of the compound of formula (I) as a free base and pharmaceutically acceptable salts thereof in the treatment of a mammal, including human, are approximately 0.01 to 250 mg/kg bodyweight at per oral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

The compound of formula (I) as a free base or a pharmaceutically acceptable salt thereof, in an essentially pure and isolated form, may be used on its own but will usually be administered in the form of a pharmaceutical formulation in which the active ingredient is in association with pharmaceutically acceptable diluents, excipients or inert carrier. Dependent on the mode of administration, the pharmaceutical formulation may comprise from 0.05 to 99% w (percent by weight), for example from 0.10 to 50% w, of active ingredient, all percentages by weight being based on total composition.

A diluent or carrier includes water, aqueous poly(ethylene glycol), magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A formulation of the invention can be in a unit dosage form such as a tablet or an injectable solution. The tablet may additionally comprise a disintegrant and/or may be coated (for example with an enteric coating or coated with a coating agent such as hydroxypropyl methylcellulose).

The invention further provides a process for the preparation of a pharmaceutical formulation of the invention which comprises mixing of the compound of formula (I) or a pharmaceutically acceptable salt thereof, a hereinbefore defined, with pharmaceutically acceptable diluents, excipients or inert carriers.

An example of a pharmaceutical formulations of the invention is an injectable solution comprising the compound of formula (I) as a free base or a pharmaceutically acceptable salt thereof, as hereinbefore defined, and sterile water, and, if necessary, either a base sodium hydroxide or an acid hydrochloric acid to bring the pH of the final formulation to about pH in the range of about 4 to 6, particularly about 5, and optionally a surfactant to aid dissolution. A suitable base is sodium hydroxide. A suitable acid is hydrochloric acid.

A suitable pharmaceutically acceptable salt of the compound of formula (I) useful in accordance to the invention is, for example, an acid-addition salt, which is sufficiently basic, for example an inorganic or organic acid. In addition a suitable pharmaceutically acceptable salt of the compounds of the invention, which is sufficiently acidic, is an alkali metal salt, an alkaline earth metal salt or a salt with an organic base, which affords a physiologically-acceptable cation.

### Medical Uses

It has been found that the compounds of formula (I) defined in the present invention, are well suited for inhibiting glycogen synthase kinase-3 (GSK3). Accordingly, said compound of the present invention is expected to be useful in the prevention and/or treatment of conditions associated with glycogen synthase kinase-3 activity, i.e. the compounds may be used to produce an inhibitory effect of GSK3 in mammals, including human, in need of such prevention and/or treatment.

GSK3 is highly expressed in the central and peripheral nervous system and in other tissues. Thus, it is expected that compound of the invention is well suited for the prevention and/or treatment of conditions associated with glycogen synthase kinase-3 in the central and peripheral nervous system. In particular, the compound of the invention is expected to be suitable for prevention and/or treatment of conditions associated with cognitive disorders and predemented states, especially dementia, Alzheimer's Disease (AD), Cognitive Deficit in Schizophrenia (CDS), Mild Cognitive Impairment (MCI), Age-Associated Memory Impairment (AAMI), Age-Related Cognitive Decline (ARCD) and Cognitive Impairement No Dementia (CIND), diseases associated with neurofibrillar tangle pathologies, Frontotemporal dementia (FTD), Frontotemporal dementia Parkinson's Type (FTDP), progressive supranuclear palsy (PSP), Pick's Disease, Niemann-Pick's Disease, corticobasal degeneration (CBD), traumatic brain injury (TBI) and dementia pugilistica.

One embodiment of the invention relates to the prevention and/or treatment of Alzheimer's Disease, especially the use in the delay of the disease progression of Alzheimer's Disease.

Other conditions are selected from the group consisting of Down's syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND, Creuztfeld-Jacob's disease and prion diseases.

Other conditions are selected from the group consisting of attention deficit disorder (ADD), attention deficit hyperactivity disorder (ADHD) and affective disorders, wherein the affective disorders are Bipolar Disorder including acute mania, bipolar depression, bipolar maintenance, major depressive disorders (MDD) including depression, major depression, mood stabilization, schizoaffective disorders including schizophrenia, and dysthymia.

Other conditions are selected from the group consisting of Type I diabetes, Type II diabetes, diabetic neuropathy, alopecia, inflammatory diseases and cancer.

One embodiment of the invention relates to the use of a compound of formula (I), as defined in the present invention, in the prevention and/or treatment of bone-related disorders or conditions in mammals.

One aspect of the invention is directed to the use of a compound of formula (I), as defined in the present invention to treat osteoporosis.

One aspect of the invention is directed to the use of a compound of formula (I), as defined in the present invention to increase and promote bone formation in mammals.

One aspect of the invention is directed to the use of a compound of formula (I), as defined in the present invention to increase bone mineral density in mammals.

Another aspect of the invention is directed to the use of a compound of formula (I), as defined in the present invention to reduce the rate of fracture and/or increase the rate of fracture healing in mammals.

Another aspect of the invention is directed to the use of a compound of formula (I), as defined in the present invention to increase cancellous bone formation and/or new bone formation in mammals.

Another aspect of the invention is directed to a method of prevention and/or treatment of bone-related disorders comprising administering to a mammal in need of such prevention and/or treatment, a therapeutically effective amount of a compound of formula (I) as defined in the present invention.

Another aspect of the invention is directed to a method of prevention and/or treatment of osteoporosis comprising administering to a mammal in need of such prevention and/or treatment, a therapeutically effective amount of a compound of formula (I) as defined in the present invention.

Another aspect of the invention is directed to a method of increasing bone formation comprising administering to a mammal in need of such treatment, a therapeutically effective amount of a compound of formula (I) as defined in the present invention.

Another aspect of the invention is directed to a method of increasing bone mineral density comprising administering to a mammal in need of such treatment, a therapeutically effective amount of a compound of formula (I) as defined in the present invention.

Another aspect of the invention is directed to a method of reducing the incidence of fracture comprising administering to a mammal in need of such treatment, a therapeutically effective amount of a compound of formula (I) as defined in the present invention.

Another aspect of the invention is directed to a method of enhancing fracture healing comprising administering to a mammal in need of such treatment, a therapeutically effective amount of a compound of formula (I) as defined in the present invention.

Another aspect of the invention is directed to said methods and wherein said mammal is a human.

Another aspect of the invention is directed to said methods and wherein said mammal is a vertibrate animal, preferably but not limited to bigger animals such as horses, camels, dromedars but not limited thereto.

The use of the GSK3 inhibitors, the compounds of formula (I) hereinbefore defined, in primary and secondary ostopeorosis, where primary osteoporosis includes postmenopausal osteoporosis and senile osteoporosis in both men and women, and secondary osteoporosis includes cortison induced osteoporosis, as well as any other type of induced secondary osteoporosis, are included in the term osteoporosis. In addition to this, these GSK3 inhibitors may also be used in treatments of myeloma. These GSK3 inhibitors may be administered locally or systemically, in different formulation regimes, to treat these conditions.

The promotion and increasing of bone formation makes the compounds of the formula (I) hereinbefore defined, suitable to reducing the incidence of fracture, to reduce the rate of fracture and/or increase the rate of fracture healing, to increase cancellous bone formation and/or new bone formation in mammals.

The use to promote and increase new bone formation may be in connection with surgery. This invention can be used during surgery, where the treating surgeon will place the invention locally in an appropriate formulation, near the deficient bone and/or in the body cavity. The bone may for instance have been broken, and utilizing the invention as described and claimed herein will then be placed in or near the fracture during open fracture repair. In some instances bone pieces may be missing (e.g. after tumour removal or severe casualties), and utilizing the invention as described and claimed herein will then be placed near the site of constructive bone surgery.

The present invention relates also to the use of the compound of formula (I) as defined in the present invention in the manufacture of a medicament for the prevention and/or treatment of conditions associated with glycogen synthase kinase-3.

The invention also provides for a method of treatment and/or prevention of conditions associated with glycogen synthase kinase-3 comprising administering to a mammal, including human in need of such treatment and/or prevention a therapeutically effective amount of the compound of formula (I) as defined in the present invention.

The dose required for the therapeutic or preventive treatment of a particular disease will necessarily be varied depending on the host treated, the route of administration and the severity of the illness being treated.

For veterinary use the amounts of different components, the dosage form and the dose of the medicament may vary and will depend on various factors such as, for example the individual requirement of the animal treated.

In the context of the present specification, the term “therapy” also includes “prevention” unless there are specific indications to the contrary. The terms “therapeutic” and “therapeutically” should be construed accordingly.

In the context of the present specification, the term “disorder” also includes “condition” unless there are specific indications to the contrary.

### Pharmacology

### Determination of ATP Competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptoethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:

MOPS Morpholinepropanesulfonic acid
EDTA Ethylenediaminetetraacetic acid

### BSA Bovin Serum Albumin

### ATP Adenosine Triphosphate

### SPA Scintillation Proximity Assay

GSK3 Glycogen synthase kinase 3

### Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

TABLE 1
Specimen results from assay.

| Example | |
|---|---|
| no | Kᵢ (nM) |
| 1 | 220 |
| 2 | 49 |
| 3 | 530 |
| 4 | 2600 |
| 5 | 28 |
| 6 | 1100 |
| 10 | 64 |
| 12 | 260 |
| 13 | 290 |
| 14 | 10 |
| 15 | 1300 |
| 16 | 75 |
| 17 | 760 |
| 18 | 42 |
| 19 | 210 |
| 20 | 37 |
| 21 | 79 |
| 22 | 150 |
| 23 | 170 |
| 24 | 230 |
| 25 | 210 |
| 26 | 75 |
| 27 | 330 |
| 28 | 280 |
| 29 | 48 |
| 30 | 200 |
| 31 | 39 |
| 33 | 210 |
| 34 | 87 |
| 35 | 120 |
| 36 | 16 |
| 37 | 140 |
| 38 | 57 |
| 39 | 36 |
| 40 | 31 |
| 41 | 120 |
| 42 | 91 |
| 43 | 53 |
| 44 | 80 |

1-84. (canceled)

85. A compound of formula (I): 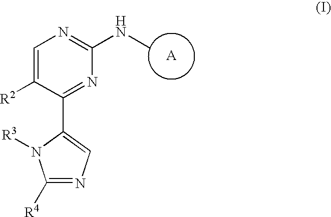
wherein:
A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by a group —R⁵—R⁷, with the proviso that said carbocyclyl is not phenyl;
R¹ is selected from halo, nitro, cyano, hydroxy, amino, sulphamoyl, carbamoyl, C₁₋₃alkyl, a carbocyclyl, a heterocyclyl and a group —R⁶—R⁷, wherein said C₁₋₃alkyl is optionally substituted by one or more halo and wherein said carbocyclyl or heterocyclyl optionally forms a conjugated ring system together with A;
R² is selected from halo, nitro, trifluoromethyl, trifluoromethoxy and cyano;
R³ is selected from methyl, C₆alkyl, C₆alkenyl, C₆alkynyl, a 6-membered non-aromatic carbocyclyl and a 6-membered non-aromatic heterocyclyl, wherein said C₆alkyl, C₆alkenyl, C₆alkynyl, carbocyclyl or heterocyclyl is optionally substituted by one or more halo, cyano, trifluoromethoxy, C₁₋₃haloalkyl or C₁₋₃alkyl;
R⁴ is selected from hydrogen, C₁₋₃alkyl, cyano and C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl;
R⁵ is selected from —C(O)N(R⁹)—, —S(O)_{Z}—, —SO₂N(R¹⁰)—, —SO₂O—, —C(O)—, —C(O)O— and (—CH₂—)ₘ; wherein R⁹ and R¹⁰ are independently selected from hydrogen or C₁₋₆alkyl and wherein said C₁₋₆alkyl is optionally substituted by one or more R¹⁹; and wherein
m is 0, 1, 2 or 3 and wherein z is 1 or 2;
R⁶ is selected from —O—, —N(R¹¹)C(O)—, —C(O)N(R¹²)—, —S(O)ᵣ—, —SO₂N(R¹³)—, —N(R¹⁴)SO₂—, —(CH₂)ₚN(R¹⁵)—, —OSO₂—, —C(O)—, —C(O)O—, —N(R¹⁶)C(O)O—, —N(R¹⁷)C(O)N(R¹⁸)—, and (—CH₂—)ₙ; wherein R¹¹, R¹², R¹³, R¹⁴, R¹⁵, R¹⁶, R¹⁷ and R¹⁸ are independently selected from hydrogen or C₁₋₆alkyl and wherein said C₁₋₆alkyl is optionally substituted by one or more R¹⁹; and wherein n is 0, 1, 2 or 3 and wherein p is 0, 1, 2 or 3 and wherein r is 0, 1 or 2;
R⁷ is selected from hydrogen, C₁₋₆alkyl, C₂₋₆alkenyl, C₂₋₆alkynyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹;
R¹⁹ and R²⁰ are independently selected from halo, nitro, cyano, hydroxy, amino, carboxy, carbamoyl, sulphamoyl, C₁₋₆alkyl, C₂₋₆alkenyl, C₂₋₆alkynyl, C₁₋₆alkoxy, C₁₋₆alkoxyC₁₋₆alkoxy, C₁₋₆alkanoyl, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R²²—, heterocyclylC₁₋₆alkyl-R²³—, carbocyclyl-R²⁴— and heterocyclyl-R²⁵—; wherein a is 0, 1 or 2; and wherein R¹⁹ and R²⁰ independently of each other is optionally substituted on carbon by one or more R²⁶; and wherein if said heterocyclyl contains an —NH— moiety that nitrogen is optionally substituted by a group selected from R²⁷;
R²², R²³, R²⁴ and R²⁵ are independently selected from —O—, —N(R²⁸)—, —C(O)—, —N(R²⁹)C(O)—, —C(O)N(R³⁰)—, —S(O)_{S}—, —SO₂N(R³¹)— and —N(R³²)SO₂—; wherein R²³, R²⁹, R³⁰, R³¹ and R³² are independently selected from hydrogen or C₁₋₆alkyl and s is 0, 1 or 2;
R²¹ and R²⁷ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, carbocyclyl, heterocyclyl, —C₁₋₆alkylcarbocylyl, —C₁₋₆alkylheterocyclyl, benzyloxycarbonyl, benzoyl and phenylsulphonyl; wherein R²¹ and R²⁷ independently of each other is optionally substituted on carbon by one or more R³³; and
R²⁶ and R³³ are independently selected from halo, nitro, cyano, —C₁₋₃alkylhydroxy, —C₁₋₃alkylmethoxy, —C₁₋₃alkylethoxy, —C₁₋₃alkylisopropoxy, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, carbocycle and heterocycle; wherein said carbocycle or heterocycle is optionally substituted by halo, methyl, trifluoromethyl, cyano or ethyl;
as a free base or a pharmaceutically acceptable salt thereof.

86. A compound according to claim 85, wherein
A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by —R⁵—R⁷, with the proviso that said carbocycle is not phenyl;
R¹ is selected from halo, nitro, cyano, hydroxy, amino, sulphamoyl, carbamoyl, C₁₋₃alkyl, a carbocyclyl, a heterocyclyl and a group —R⁶—R⁷, wherein said C₁₋₃alkyl is optionally substituted by one or more halo and wherein said carbocyclyl or heterocyclyl optionally forms a conjugated ring system together with A;
R² is selected from halo, trifluoromethyl, trifluoromethoxy and cyano;
R³ is selected from methyl, C₆alkyl, C₆alkenyl, C₆alkynyl, a 6-membered non-aromatic carbocyclyl and a 6-membered non-aromatic heterocyclyl, wherein said C₆alkyl, C₆alkenyl, C₆alkynyl, carbocyclyl or heterocyclyl is optionally substituted by one or more halo, cyano, trifluoromethoxy, C₁₋₃haloalkyl or C₁₋₃alkyl;
R⁴ is selected from hydrogen, C₁₋₃alkyl, cyano and C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl;
R⁵ is selected from —C(O)N(R⁹)—, —S(O)_{Z}—, —SO₂N(R¹⁰)—, —SO₂O—, —C(O)—, —C(O)O— and (—CH₂—)ₘ; wherein R⁹ and R¹⁰ are independently selected from hydrogen or C₁₋₆alkyl and wherein said C₁₋₆alkyl is optionally substituted by one or more R¹⁹; and wherein
m is 0, 1, 2 or 3 and wherein z is 1 or 2;
R⁶ is selected from —O—, —N(R¹¹)C(O)—, —C(O)N(R¹²)—, —S(O)ᵣ—, —SO₂N(R¹³)—, —N(R¹⁴)SO₂—, —(CH₂)ₚN(R¹⁵)—, —OSO₂—, —C(O)—, —C(O)O—, —N(R¹⁶)C(O)O—, —N(R¹⁷)C(O)N(R¹⁸)—, and (—CH₂—)ₙ; wherein R¹¹, R¹², R¹³, R¹⁴, R¹⁵, R¹⁶, R¹⁷ and R¹⁸ are independently selected from hydrogen or C₁₋₆alkyl and wherein said C₁₋₆alkyl is optionally substituted by one or more R¹⁹; wherein n is 0, 1, 2 or 3 and wherein p is 0, 1, 2 or 3 and wherein r is 0, 1 or 2;
R⁷ is selected from hydrogen, C₁₋₆alkyl, C₂₋₆alkenyl, C₂₋₆alkynyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹;
R¹⁹ and R²⁰ are independently selected from halo, nitro, cyano, hydroxy, amino, carboxy, C₁₋₆alkyl, C₂₋₆alkenyl, C₂₋₆alkynyl, C₁₋₆alkoxy, C₁₋₆alkoxyC₁₋₆alkoxy, C₁₋₆alkanoyl, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ, carbocyclyl, heterocyclyl, carbocyclylC₁₋₆alkyl-R²²—, heterocyclylC₁₋₆alkyl-R²³—, carbocyclyl-R²⁴— and heterocyclyl-R²⁵—; wherein a is 0, 1 or 2; and wherein R¹⁹ and R²⁰ independently of each other is optionally substituted on carbon by one or more R²⁶; and wherein if said heterocyclyl contains an —NH— moiety that nitrogen is optionally substituted by a group selected from R²⁷;
R²², R²³, R²⁴ and R²⁵ are independently selected from —O—, —N(R²⁸)—, —C(O)—, —N(R²⁹)C(O)—, —C(O)N(R³⁰)—, —S(O)_{S}—, —SO₂N(R³¹)— and —N(R³²)SO₂—; wherein R²³, R²⁹, R³⁰, R³¹ and R³² are independently selected from hydrogen or C₁₋₆alkyl and s is 0, 1 or 2;
R²¹ and R²⁷ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, carbocyclyl, heterocyclyl, —C₁₋₆alkylcarbocylyl, —C₁₋₆alkylheterocyclyl, benzyloxycarbonyl, benzoyl and phenylsulphonyl; wherein R²¹ and R²⁷ independently of each other is optionally substituted on carbon by one or more R³³; and
R²⁶ and R³³ are independently selected from halo, nitro, cyano, —C₁₋₃alkylhydroxy, —C₁₋₃alkylmethoxy, —C₁₋₃alkylethoxy, —C₁₋₃alkylisopropoxy, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, methylthio, ethylthio, methylsulphinyl, ethylsulphinyl, mesyl, ethylsulphonyl, methoxycarbonyl, ethoxycarbonyl, N,N-dimethylsulphamoyl, N,N-diethylsulphamoyl, N-methyl-N-ethylsulphamoyl, carbocycle and heterocycle;
wherein said carbocycle or heterocycle is optionally substituted by halo, methyl, trifluoromethyl, cyano or ethyl.

87. A compound according to claim 85 or 86, wherein
A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by —R⁵—R⁷ with the proviso that said carbocyclyl is not phenyl;
R¹ is selected from C₁₋₃alkyl, a carbocyclyl, a heterocyclyl and a group —R⁵—R⁷, wherein said C₁₋₃alkyl is optionally substituted by one or more halo and wherein said carbocyclyl or heterocyclyl optionally forms a conjugated ring system together with A;
R² is selected from halo, trifluoromethyl, trifluoromethoxy and cyano;
R³ is selected from methyl, C₆alkyl, a 6-membered non-aromatic carbocyclyl and a 6-membered non-aromatic heterocyclyl, wherein said C₆alkyl, carbocyclyl or heterocyclyl is optionally substituted by one or more halo, cyano, trifluoromethoxy, C₁₋₃haloalkyl or C₁₋₃alkyl;
R⁴ is selected from hydrogen, C₁₋₃alkyl, cyano and C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl;
R⁵ is selected from —C(O)N(R⁹)—, —S(O)_{Z}—, —SO₂N(R¹¹)—, —SO₂O—, —C(O)—, —C(O)O— and (—CH₂—)ₘ; wherein R⁹ and R¹⁰ are independently selected from hydrogen or C₁₋₆alkyl and wherein said C₁₋₆alkyl is optionally substituted by one or more R¹⁹; and wherein
m is 0, 1, 2 or 3 and wherein z is 1 or 2;
R⁶ is selected from —O—, —N(R¹¹)C(O)—, —C(O)N(R¹²)—, —S(O)ᵣ—, —SO₂N(R¹³)—, —N(R¹⁴)SO₂—, —(CH₂)ₚN(R¹⁵)—, —OSO₂—, —C(O)—, —C(O)O—, —N(R¹⁶)C(O)O—, —N(R¹⁷)C(O)N(R¹⁸)—, and (—CH₂—)ₙ; wherein R¹¹, R¹², R¹³, R¹⁴, R¹⁵, R¹⁶, R¹⁷ and R¹⁸ are independently selected from hydrogen or C₁₋₆alkyl and wherein said C₁₋₆alkyl is optionally substituted by one or more R¹⁹; and wherein n is 0, 1, 2 or 3 and wherein p is 0, 1, 2 or 3 and wherein r is 0, 1 or 2;
R⁷ is selected from hydrogen, C₁₋₆alkyl, C₂₋₆alkenyl, C₂₋₆alkynyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹;
R¹⁹ and R²⁰ are independently selected from halo, nitro, cyano, hydroxy, amino, carboxy, C₁₋₆alkyl, C₂₋₆alkenyl, C₂₋₆alkynyl, C₁₋₆alkoxy, C₁₋₆alkoxyC₁₋₆alkoxy, C₁₋₆alkanoyl, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, carbocyclyl, heterocyclyl, carbocyclylC₁₋₆alkyl-R²²—, heterocyclylC₁₋₆alkyl-R²³—, carbocyclyl-R²⁴— and heterocyclyl-R²⁵—; and wherein R¹⁹ and R²⁰ independently of each other is optionally substituted on carbon by one or more R²⁶; and wherein if said heterocyclyl contains an —NH— moiety that nitrogen is optionally substituted by a group selected from R²⁷;
R²², R²³, R²⁴ and R²⁵ are independently selected from —O—, —N(R²⁸)—, —C(O)—, —N(R²⁹)C(O)—, —C(O)N(R³⁰)—, —S(O)_{S}—, —SO₂N(R³¹)— and —N(R³²)SO₂—; wherein R²³, R²⁹, R³⁰, R³¹ and R³² are independently selected from hydrogen or C₁₋₆alkyl and s is 0, 1 or 2;
R²¹ and R²⁷ are independently selected from C₁₋₆alkyl, C₁₋₆alkanoyl, C₁₋₆alkoxycarbonyl, carbamoyl, N—(C₁₋₆alkyl)carbamoyl, N,N—(C₁₋₆alkyl)carbamoyl, carbocyclyl, heterocyclyl, —C₁₋₆alkylcarbocylyl, —C₁₋₆alkylheterocyclyl, benzoyl and phenylsulphonyl; wherein R²¹ and R²⁷ independently of each other is optionally substituted on carbon by one or more R³³; and
R²⁶ and R³³ are independently selected from halo, nitro, cyano, —C₁₋₃alkylhydroxy, —C₁₋₃alkylmethoxy, —C₁₋₃alkylethoxy, —C₁₋₃alkylisopropoxy, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, methylthio, ethylthio, methylsulphinyl, mesyl, ethylsulphonyl, methoxycarbonyl, ethoxycarbonyl, N,N-diethylsulphamoylcarbocycle and heterocycle; wherein said carbocycle or heterocycle is optionally substituted by halo, methyl, trifluoromethyl, cyano or ethyl.

88. A compound according to claim 85, wherein R² is halo or cyano.

89. A compound according to claim 85, wherein R² is halo.

90. A compound according to claim 89, wherein R² is fluoro.

91. A compound according to claim 85, wherein R³ is selected from a 6-membered non-aromatic carbocyclyl or a 6-membered non-aromatic heterocyclyl, wherein said carbocyclyl or heterocyclyl is optionally substituted by one or more halo, cyano, trifluoromethoxy, C₁₋₃haloalkyl or C₁₋₃alkyl.

92. A compound according to claim 90, wherein R³ is selected from a 6-membered non-aromatic carbocyclyl or a 6-membered non-aromatic heterocyclyl, wherein said carbocyclyl or heterocyclyl is optionally substituted by one or more halo, cyano, trifluoromethoxy, C₁₋₃haloalkyl or C₁₋₃alkyl.

93. A compound according to claim 85 or claim 92, wherein R³ is a non-aromatic 6-membered heterocyclyl.

94. A compound according to claim 85 or claim 92, wherein R³ is 3-tetrahydropyranyl or 4-tetrahydropyranyl.

95. A compound according to claim 85 or claim 92, wherein R³ is 4-tetrahydropyranyl.

96. A compound according to claim 85 or claim 92, wherein R⁴ is C₁₋₃alkyl or C₁₋₃haloalkyl, wherein said C₁₋₃alkyl or C₁₋₃haloalkyl is optionally substituted with one or more OR⁸; wherein R⁸ is independently selected from hydrogen, C₁₋₆alkyl or C₁₋₆haloalkyl.

97. A compound according to claim 85 or claim 92, wherein R⁴ is C₁₋₃alkyl.

98. A compound according to claim 85 or claim 92, wherein R⁴ is methyl.

99. A compound according to claim 85 or claim 92, wherein A is heterocyclyl; wherein said heterocyclyl is optionally substituted on carbon by one or more R¹ and wherein if said heterocyclyl contains an —NH— moiety that nitrogen may be optionally substituted by —R⁵—R⁷.

100. A compound according to claim 98, wherein A is 4-piperidinyl, 4-tetrahydropyranyl, 3-pyridyl, 4-pyridyl, 5-pyrimidinyl, 4-isoquinolinyl or 2-pyridyl.

101. A compound according to claim 85 or claim 92, wherein A is a non-aromatic carbocyclyl; wherein said carbocyclyl is optionally substituted on carbon by one or more R¹.

102. A compound according to claim 101, wherein said non-aromatic carbocyclyl is cyclohexyl.

103. A compound according to claim 85 or claim 92, wherein R¹ is C₁₋₃alkyl, wherein said C₁₋₃alkyl may be optionally substituted by one or more halo.

104. A compound according to claim 103, wherein R¹ is methyl.

105. A compound according to claim 103, wherein R¹ is C₁₋₃alkyl substituted by one or more halo.

106. A compound according to claim 105, wherein R¹ is trifluoromethyl.

107. A compound according to claim 85 or claim 92, wherein R¹ is selected from a group —R⁶—R⁷.

108. A compound according to claim 107, wherein R⁶ is selected from —O—, —(CH₂)ₚN(R¹⁵)—, —C(O)—, —C(O)O—, —N(R¹⁶)C(O)O—, and (—CH₂—)ₙ.

109. A compound according to claim 108, wherein R⁶ is selected from —O—, —(CH₂)ₚN(R¹⁵)—, —C(O)— and (—CH₂—)ₙ.

110. A compound according to claim 108, wherein R⁶ is (—CH₂—)ₙ and n is 0 or 1.

111. A compound according to claim 109, wherein R⁶ is (—CH₂—)ₙ and n is 0 or 1.

112. A compound according to claim 108, wherein R⁶ is —(CH₂)ₚN(R¹⁵)— and p is 1.

113. A compound according to claim 109, wherein R⁶ is —(CH₂)ₚN(R¹⁵)— and p is 1.

114. A compound according to claim 85 or claim 92, wherein R⁵ is selected from —C(O)N(R⁹)—, —S(O)_{Z}—, —C(O)—, —C(O)O— and (—CH₂—)ₘ; and wherein m is 0 or 1 and wherein z is 2.

115. A compound according to claim 114, wherein R⁵ is selected from, —S(O)_{Z}—, —C(O)—, —C(O)O— and (—CH₂—)ₘ; and wherein m is 0 or 1 and wherein z is 2.

116. A compound according to claim 85 or claim 92, wherein R⁷ is selected from hydrogen, C₁₋₆alkyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹.

117. A compound according to claim 107, wherein R⁷ is selected from hydrogen, C₁₋₆alkyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹.

118. A compound according to claim 116, wherein R⁷ is C₁₋₆alkyl, heterocyclyl or carbocyclyl; wherein R⁷ may be optionally substituted on carbon by one or more R²⁰; and wherein if said heterocyclyl contains an —NH— moiety that nitrogen may be optionally substituted by a group selected from R²¹.

119. A compound according to claim 117, wherein R⁷ is C₁₋₆alkyl, heterocyclyl or carbocyclyl; wherein R⁷ may be optionally substituted on carbon by one or more R²⁰; and wherein if said heterocyclyl contains an —NH— moiety that nitrogen may be optionally substituted by a group selected from R²¹.

120. A compound according to claim 117 or 118, wherein R⁷ is C₁₋₆alkyl.

121. A compound according to claim 117 or 118, wherein R⁷ is methyl.

122. A compound according to claim 98 or claim 99, wherein A is not substituted.

123. A compound according to claim 85, wherein
A is heterocyclyl or carbocyclyl; wherein said heterocyclyl or carbocyclyl is optionally substituted on carbon by one or more R¹ and wherein if said heterocyclyl contains an —NH— moiety that nitrogen may be optionally substituted by a group —R⁵—R⁷, with the proviso that said carbocyclyl is not phenyl;
R¹ is selected from C₁₋₃alkyl, a carbocyclyl, and a group —R⁶—R⁷, wherein said C₁₋₃alkyl is optionally substituted by one or more halo;
R² is halo;
R³ is a 6-membered non-aromatic heterocyclyl;
R⁴ is C₁₋₃alkyl;
R⁵ is selected from —S(O)_{Z}—, —C(O)—, —C(O)O— and (—CH₂—)ₘ; and wherein m is 0 or 1 and wherein z is 2;
R⁶ is selected from —O—, —(CH₂)ₚN(R¹⁵)—, —C(O)—, and (—CH₂—)ₙ; wherein R¹⁵ is selected from hydrogen or C₁₋₆alkyl and wherein said C₁₋₆alkyl is optionally substituted by one or more R¹⁹; and wherein n is 0 or 1 and wherein p is 1;
R⁷ is selected from hydrogen, C₁₋₆alkyl, —C₁₋₄alkylcarbocyclyl, —C₁₋₄alkylheterocyclyl, carbocyclyl and heterocyclyl; wherein R⁷ may be optionally substituted on carbon by one or more R²⁰; and wherein if said heterocyclyl contains an —NH— moiety that nitrogen may be optionally substituted by a group selected from R²¹;
R¹⁹ and R²⁰ are independently selected from halo, cyano, C₁₋₆alkyl, C₁₋₆alkoxy, N—(C₁₋₆alkyl)amino, N,N—(C₁₋₆alkyl)₂amino, carbocyclyl and heterocyclyl; and wherein R¹⁹ and R²⁰ independently of each other is optionally substituted on carbon by one or more R²⁶;
R²¹ is C₁₋₆alkanoyl or heterocyclyl; and
R²⁶ is selected from halo, cyano, —C₁₋₃alkylmethoxy, hydroxy, methyl, heterocycle and methoxy; wherein said carbocycle or heterocycle is optionally substituted by halo.

124. A compound according to claim 123, wherein R² is fluoro.

125. A compound according to claim 123, wherein R³ is 4-tetrahydropyranyl.

126. A compound according to claim 123, wherein R⁴ is methyl.

127. A compound according to claim 85, wherein
A is heterocyclyl wherein said heterocyclyl is optionally substituted, on carbon, by one or more R¹;
R¹ is C₁₋₃alkyl or a group —R⁶—R⁷, wherein said C₁₋₃alkyl may be optionally substituted by one or more halo;
R² is halo;
R³ is a 6-membered non-aromatic heterocyclyl;
R⁴ is C₁₋₃alkyl;
R⁶ is —O—, or —C(O)—; and
R⁷ is C₁₋₆alkyl.

128. A pharmaceutical formulation comprising as active ingredient a therapeutically effective amount of a compound according to claim 85 in association with pharmaceutically acceptable excipients, carriers or diluents.

129. A method of prevention and/or treatment of conditions associated with glycogen synthase kinase-3, comprising administering to a human in need of such prevention and/or treatment a therapeutically effective amount of a compound salt as defined in claim 85.

130. A method of prevention and/or treatment of cognitive disorders, comprising administering to a human in need of such prevention and/or treatment a therapeutically effective amount of a salt compound as defined in claim 85.

131. The method according to claim 130, wherein the cognitive disorder is dementia, Cognitive Deficit in Schizophrenia (CDS), Mild Cognitive Impairment (MCI), Age-Associated Memory Impairment (AAMI), Age-Related Cognitive Decline (ARCD) or Cognitive Impairement No Dementia (CIND).

132. The method according to claim 131, wherein the disease is Cognitive Deficit in Schizophrenia.

133. The method according to claim 131, wherein the dementia is associated with neurofibrillar tangle pathologies.

134. The method according to claim 131, wherein the dementia is Frontotemporal dementia (FTD), Frontotemporal dementia Parkinson's Type (FTDP), progressive supranuclear palsy (PSP), Pick's Disease, Niemann-Pick's Disease, corticobasal degeneration, traumatic brain injury (TBI) or dementia pugilistica.

135. The method according to claim 131, wherein the dementia is Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Creuztfeld-Jacob's disease or prion diseases.

136. The method according to claim 135, wherein the dementia is Alzheimer's Disease.

137. The method according to claim 135, wherein the treatment is in the delay of the disease progression of Alzheimer's Disease.

138. A method of prevention and/or treatment of attention deficit disorder (ADD), attention deficit hyperactivity disorder (ADHD) or affective disorders, comprising administering to a human in need of such prevention and/or treatment a therapeutically effective amount of a compound salt as defined in claim 85.

139. The method according to claim 138, wherein the affective disorders are Bipolar Disorder including acute mania, bipolar depression, bipolar maintenance, major depressive disorders (MDD) including depression, major depression, mood stabilization, schizoaffective disorders including schizophrenia, or dysthymia.

140. A method of prevention and/or treatment of Type I diabetes, Type II diabetes, diabetic neuropathy, alopecia, inflammatory diseases or cancer, comprising administering to a human in need of such prevention and/or treatment a therapeutically effective amount of a salt compound as defined in claim 85.

141. A method of prevention and/or treatment of bone related disorders or conditions comprising administering to a human in need of such prevention and/or treatment a therapeutically effective amount of a salt compound as defined in claim 85.

142. A method of prevention and/or treatment of osteoporosis comprising administering to a human in need of such prevention and/or treatment a therapeutically effective amount of a compound as defined in claim 85.

143. A method of increasing bone formation comprising administering to a human in need of such prevention and/or treatment a therapeutically effective amount of a compound as defined in claim 85.

144. A method of increasing cancellous bone formation and/or new bone formation comprising administering to a human in need of such prevention and/or treatment a therapeutically effective amount of a compound as defined in claim 85.

145. A method of increasing bone mineral density comprising administering to a human in need of such prevention and/or treatment a therapeutically effective amount of a compound as defined in claim 85.

146. A method of reducing the incidence of fracture comprising administering to a human in need of such prevention and/or treatment a therapeutically effective amount of a compound as defined in claim 85.

147. A method of enhancing fracture healing comprising administering to a human in need of such prevention and/or treatment a therapeutically effective amount of a compound as defined in claim 85.

148. A process for preparing a compound of formula (I) or a pharmaceutically acceptable salt or an in vivo hydrolysable ester thereof comprising the following steps:
a) reacting a pyrimidine of formula (II): 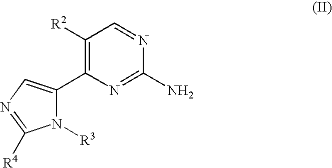
with a compound of formula (III): 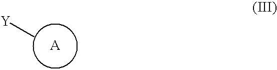
wherein R¹, R², R³, R⁴ and A are, unless otherwise specified, as defined in claim 85;
wherein A contains an aromatic mono- or bicyclic heterocycle;
wherein Y is a displaceable group;
and thereafter optionally:
b) converting a compound of formula (I) into another compound of formula (I);
c) removing any protecting groups; and
d) forming a pharmaceutically acceptable salt or in vivo hydrolysable ester.
